(19) United States
(12) Patent Application Publication
(10) Pub. No.: US 2012/0015998 A1
(43) Pub. Date: Jan. 19, 2012

(54) PHOTOTHERAPY METHODS AND DEVICES COMPRISING EMISSIVE ARYL-HETEROARYL COMPOUNDS

(75) Inventors: Amane Mochizuki, San Diego, CA (US); Sazzadur Rahman Khan, San Diego, CA (US); Sheng Li, Vista, CA (US); Shijun Zheng, San Diego, CA (US); Jensen Cayas, Bonita, CA (US); Keisaku Okada, San Diego, CA (US); Brett T. Harding, Carlsbad, CA (US)

(73) Assignee: Nitto Denko Corporation, Osaka (JP)

(21) Appl. No.: 13/179,826

(22) Filed: Jul. 11, 2011

Related U.S. Application Data
(60) Provisional application No. 61/363,780, filed on Jul. 13, 2010.

(51) Int. Cl. A61K 31/409; C07D 403/12; C07D 263/57; A61K 31/221; A61K 31/197; A61P 33/02; A61P 17/00; A61P 35/00; A61P 27/02; A61P 29/00; A61P 3/04; A61P 9/00; C07D 235/18; A61P 31/00

(52) U.S. Cl. 514/410; 548/309.7; 548/305.1; 548/224; 514/551; 514/561

(57) ABSTRACT

Disclosed herein are compounds represented by a formula: R¹—Ar¹—X—Ar²—Ar³-Het, wherein R¹, Ar¹, X, Ar², Ar³, and Het are described herein. Compositions and light-emitting devices related thereto are also disclosed.

### CROSS-REFERENCE TO RELATED APPLICATIONS

This application claims the benefit of U.S. Provisional Application No. 61/363,780, filed Jul. 13, 2010, which is incorporated by reference herein in its entirety.

### BACKGROUND

1. Field of the Invention

This invention relates to light-emitting compounds and compositions, as well as light-emitting devices that include the light-emitting compounds or compositions.

2. Description of the Related Art

Phototherapy may be useful in treating a number of medical conditions. However, light sources such as lasers, which may be used for phototherapy, may be expensive, difficult to transport, and not suitable for home or outpatient treatment. Therefore, there is a need for alternative sources of light for phototherapy which may be less expensive and more portable.

### SUMMARY OF THE INVENTION

Some embodiments relate to organic light-emitting devices which may be used for phototherapy. These devices typically comprise an organic light-emitting which layer may comprise a light-emitting component, such as a compound described herein. For example, some of the compounds described herein may be useful as deep blue emitters. Some embodiments provide compounds which comprise a series of 2, 3, or 4 aryl rings which may be directly connected or be interrupted by 1 or 2 oxygen atoms.

Some embodiments provide a light-emitting device for use in phototherapy comprising: a light-emitting layer comprising a compound represented by Formula 1:

R¹—Ar¹—X—Ar²—Ar³-Het (Formula 1)

wherein R¹ is a C₁-10O₁₋₄ ether attaching at an oxygen atom or —R⁷—NR⁸R⁹; wherein R⁷ is a single bond, optionally substituted C₆₋₁₀ aryloxy, or optionally substituted C₆₋₁₀ aryl; and R⁸ and R⁹ are independently optionally substituted C₆₋₁₀ aryl, wherein R⁸ and R⁹ optionally link aryl; X is O or a single bond; Ar³ is optionally substituted aryl; or Ar³ is a single bond; and Het is optionally substituted heteroaryl, including C₆₋₁₀ heteroaryl such as optionally substituted benzooxazolyl, optionally substituted benzothiazolyl, or optionally substituted benzoimidazolyl; and wherein the device is configured to emit a therapeutically effective amount of light to a mammal.

In some embodiments, a light-emitting device for use in phototherapy comprising: a light-emitting layer comprises a compound represented by Formula 2:

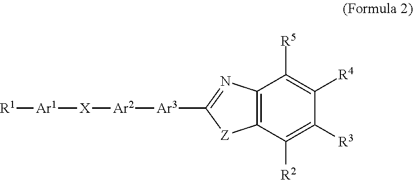

wherein R¹, Ar¹, Ar², Ar³, and X are the same as described for Formula 1; Z is independently NR⁶, O, or S, wherein R⁶ is optionally substituted phenyl, optionally substituted —CH₂-phenyl, or optionally substituted (4-halophenyl)methyl; and R², R³, R⁴, and R⁵ are independently H, optionally substituted C₆₋₃₀ aryl, C₁₋₁₀ alkyl, or C₁₋₁₀ alkoxy.

In some embodiments, these devices may be used in a method of carrying out phototherapy comprising: exposing at least a portion of a tissue of a mammal to light from a device described herein. In some embodiments, the tissue comprises a photosensitive compound which is not naturally in the tissue, and at least a portion of the photosensitive compound is activated by exposing the portion of the tissue to light from the device.

Some embodiments provide a method of treating a disease, comprising: administering a photosensitive compound to a tissue of a mammal in need thereof; exposing at least a portion of the tissue to light from a device described herein; and wherein at least a portion of the photosensitive compound is activated by at least a portion of the light from the device to which the tissue is exposed, to thereby treat the disease.

Some embodiments provide a phototherapy system comprising: a device described herein; and a photosensitive compound; wherein the photosensitive compound is suitable for administration to a tissue of a mammal in need of phototherapy; and wherein the device is configured to emit light of a wavelength which can activate at least a portion of the photosensitive compound when it is in the tissue.

These and other embodiments are described in greater detail below.

### BRIEF DESCRIPTION OF THE DRAWINGS

FIG. 1 is a schematic of an embodiment of a light-emitting device suitable for phototherapy which comprises a controller and a processor.

FIG. 2 shows an embodiment of an organic light-emitting device incorporating a compound of Formula 1.

FIG. 3 is a graph depicting the electroluminescence spectrum of an embodiment of an organic light-emitting device of FIG. 2.

FIG. 4 is a graph depicting the current density (mA/cm2) and brightness (cd/m2) as a function of driving voltage of an embodiment of an organic light-emitting device of FIG. 2.

FIG. 5 is a graph depicting the External Quantum Efficiency (EQE) as a function of current density of an embodiment of an organic light-emitting device of FIG. 2.

FIG. 6 is a graph depicting the luminous efficiency (cd/A) and power efficiency (lm/W) as a function of current density (mA/cm2) of an embodiment of an organic light-emitting device of FIG. 2.

FIG. 7 shows a graph depicting the power output (mW/cm2) as a function of driving voltage of an embodiment of an organic light-emitting device of FIG. 2.

FIG. 8 is a schematic representation of ex-vivo efficacy study with the device output according to FIG. 7.

FIG. 9 shows the image of the cells before and after the light irradiation from OLED.

FIG. 10 is shows cell viability (%) data after irradiating 25 J/cm2 with different concentration of photo sensitizer.

FIG. 11 shows the cell viability data with 1 mM photosensitizer and after irradiating different doses (J/cm2) of light from OLED.

### DETAILED DESCRIPTION

Unless otherwise indicated, when a chemical structural feature such as alkyl or aryl is referred to as being “optionally substituted,” it is meant that the feature may have no substituents (i.e. be unsubstituted) or may have one or more substituents. A feature that is “substituted” has one or more substituents. The term “substituent” has the ordinary meaning known to one of ordinary skill in the art. In some embodiments, the substituent is a halogen, or has from 1-20 carbon atoms, from 1-10 carbon atoms, or has a molecular weight of less than about 500 g/mol, about 300 g/mol, or about 200 g/mol. In some embodiments, the substituent has at least 1 carbon atom or at least 1 heteroatom, and has about 0-10 carbon atoms and about 0-5 heteroatoms independently selected from: N, O, S, F, Cl, Br, I, and combinations thereof. In some embodiments, each substituent consists of about 0-20 carbon atoms, about 0-47 hydrogen atoms, about 0-5 oxygen atoms, about 0-2 sulfur atoms, about 0-3 nitrogen atoms, about 0-1 silicon atoms, about 0-7 fluorine atoms, about 0-3 chlorine atoms, about 0-3 bromine atoms, and about 0-3 iodine atoms. Examples include, but are not limited to, alkyl, alkenyl, alkynyl, carbazolyl, cycloalkyl, cycloalkenyl, cycloalkynyl, aryl, diarylamino, heteroaryl, heteroalicyclyl, aralkyl, heteroaralkyl, (heteroalicyclyl)alkyl, hydroxy, protected hydroxy, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxyl, trihalomethanesulfonyl, trihalomethanesulfonamido, and amino, including mono- and di-substituted amino groups, and the protected derivatives thereof. In some embodiments, the substituent may be selected from F, Cl, Br, I, NO₂, —CN, —CNO, —NCO, R′, —OR′, —COR′, —CO₂R′, —OCOR′, —NR′COR″, CONR′R″, —NR′R″, wherein each R′ and R″ is independently H, optionally substituted phenyl, C₁₋₁₂ alkyl, or C₁₋₆ alkyl. In some embodiments, the substituent may be selected from R′, —OR′, —NR′R″, wherein each R′ and R″ is independently H, optionally substituted phenyl, C₁₋₁₂ alkyl, or C₁₋₆ alkyl.

The term “electron-donating substituent” has the ordinary meaning known to one of ordinary skill in the art. In some embodiments, the electron-donating substituent is a halogen, or has about 1-20 carbon atoms, about 1-10 carbon atoms, or has a molecular weight of less than about 500, about 300, or about 200. In some embodiments, the electron-donating substituent has at least 1 carbon atom or at least 1 heteroatom, and has about 0-10 carbon atoms and about 0-5 heteroatoms independently selected from: N, O, S, and combinations thereof. In some embodiments, the electron-donating substituent is an electron donor with respect to a phenyl ring to which it is attached. Some examples of electron-donating substituents may include, but are not limited to: alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxyl, aryloxy, O-ester, mercapto, alkylthio, arylthio, O-carbamyl, N-carbamyl, O-thiocarbamyl, N-thiocarbamyl, N-amido, O-carboxy, silyl, and amino.

The term “electron-withdrawing substituent” has the ordinary meaning known to one of ordinary skill in the art. In some embodiments, the electron-withdrawing substituent is a halogen, or has about 1-20 carbon atoms, about 1-10 carbon atoms, or has a molecular weight of less than about 500, about 300, or about 200. In some embodiments, the electron-donating substituent has at least 1 carbon atom or at least 1 heteroatom, and has about 0-10 carbon atoms and about 0-5 heteroatoms independently selected from: N, O, S, F, Cl, and combinations thereof. In some embodiments, the electron-withdrawing substituent is electron withdrawing with respect to a phenyl ring to which it is attached. Some examples of electron-withdrawing substituents may include, but are not limited to: acyl, C-ester, cyano, F, Cl, carbonyl, C-amido, thiocarbonyl, C-carboxy, protected C-carboxy, isocyanato, thiocyanato, isothiocyanato, nitro, sulfinyl, sulfonyl, perflouoralkyl, trihalomethanesulfonyl, and trihalomethanesulfonamido.

The term “aryl” as used herein refers to an aromatic ring or ring system. Exemplary non-limiting aryl groups are phenyl, naphthyl, etc. “C_{x-y} aryl” refers to aryl where the ring or ring system has x-y carbon atoms. The indicated number of carbon atoms for the ring or ring system does not include or limit the number of carbon atoms in any substituents attached to the ring or ring system. Examples include, but are not limited to, optionally substituted phenyl, optionally substituted naphthyl, optionally substituted anthracenyl, optionally substituted p-interphenylene, optionally substituted 1,4-internaphthylene, and optionally substituted 9,10-interanthracenylene. These are shown below in their unsubstituted forms. However, any carbon not attached to the remainder of the molecule may optionally have a substituent.

The term “heteroaryl” refers to “aryl” which has one or more heteroatoms in the ring or ring system. “C_{x-y} heteroaryl” refers to heteroaryl where the ring or ring system has x-y carbon atoms. The indicated number of carbon atoms for the ring or ring system does not include or limit the number of carbon atoms in any substituents attached to the ring or ring system. Examples of “heteroaryl” may include, but are not limited to, pyridinyl, furyl, thienyl, oxazolyl, thiazolyl, imidazolyl, indolyl, quinolinyl, benzofuranyl, benzothienyl, benzooxazolyl, benzothiazolyl, benzoimidazolyl, etc.

The term “diarylamino” as used herein refers to a moiety comprising a nitrogen atom which attaches to the remainder of the molecule (e.g. Ar¹), and the nitrogen atom is also directly attached to two optionally substituted aryl groups, the term aryl being described above. “C_{x-y} diarylamino” as used herein refers a total number of carbon atoms in the range x-y in the two aryl rings. The indicated number of carbon atoms for the aryl rings does not include or limit the number of carbon atoms in any substituents attached to the ring or ring system. Examples include, but are not limited to, diphenyl amine (such as unsubstituted diphenyl amine or substituted diphenyl amine, e.g. phenyl(methylphenyl)amine, ditolyl amine), etc.

The term “diarylaminophenoxy” as used herein refers to an optionally substituted phenoxy moiety (i.e. optionally substituted —O-phenyl), wherein the phenyl has an optionally substituted diarylamino substituent. “C_{x-y} diarylaminophenoxy” as used herein refers a total number of carbon atoms in the range of x-y in the two aryl rings and in the phenyl ring. The indicated number of carbon atoms for the aryl rings does not include or limit the number of carbon atoms in any substituents attached to the ring or ring system. Examples include, but are not limited to, p-diphenylaminophenoxy (such as unsubstituted p-diphenylaminophenoxy, or p-diphenylaminophenoxy substituted with 1, 2, 3, or 4 methyl substituents, etc.).

The names for several moieties used herein are indicated with the corresponding structures below. For any of these moieties, any carbon atom not attached to the remainder of the molecule, or any NH nitrogen, may optionally have a substituent.

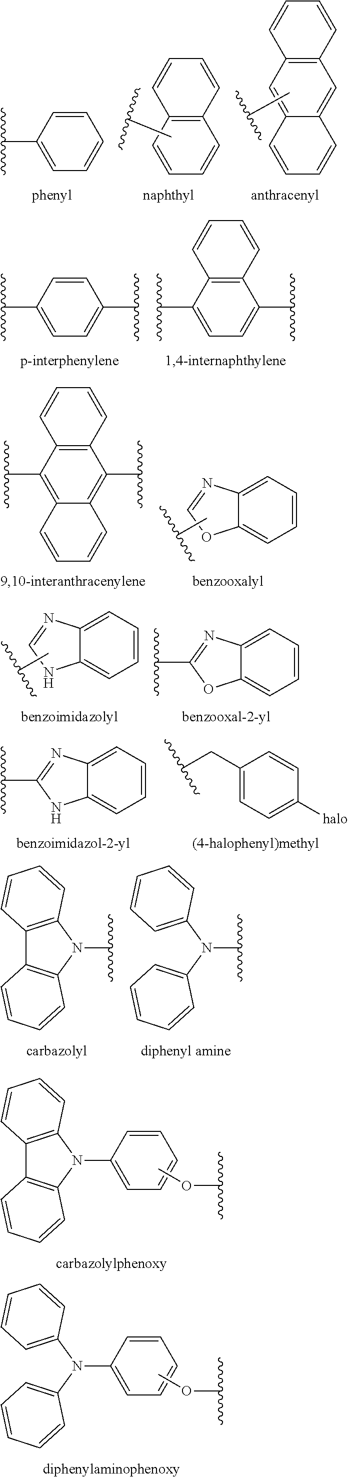

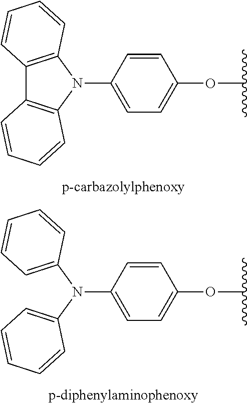

The term “alkyl” as used herein refers to a moiety comprising carbon and hydrogen containing no double or triple bonds. Alkyl may be linear, branched, cyclic, or a combination thereof, and contain from one to thirty-five carbon atoms. Examples of alkyl groups include but are not limited to methyl, ethyl, propyl, isopropyl, cyclopropyl, n-butyl, iso-butyl, tert-butyl, cyclobutyl, pentyl isomers, cyclopentane, hexyl isomer, cyclohexane, and the like. The term “linear alkyl” as used herein refers to —(CH₂)_{q}CH₃, where q is 0-34. The term “C₁₋₁₀ alkyl” as used herein refers to alkyl having from 1 to 10 carbon atoms such as methyl, ethyl, propyl isomers, butyl isomers, cyclobutyl isomers, pentyl isomers, cyclopentyl isomers, hexyl isomers, cyclohexyl isomer, heptyl isomers, cycloheptyl isomers, octyl isomers, cyclooctyl isomers, nonyl isomers, cyclononyl isomers, decyl isomer, cyclodecyl isomers, etc. The term “alkylene” is a subgenus of “alkyl” and refers to a divalent alkyl moiety, e.g. —CH₂—, etc.

The term “ether” as used herein refers to a moiety comprising carbon, hydrogen, and single bonded oxygen, i.e. —O—, provided that —O—O— is not present. The phrase “C₁₋₁₀O₁₋₄ ether” refers to ether having from 1-10 carbon atoms and 1-4 oxygen atoms. The phrase “attaching at an oxygen atom” refers to a situation where the atom of the ether moiety which attaches to the rest of the structure (e.g. Ar¹) is an oxygen atom. Examples include alkoxy, polyalkylene oxide, etc. The term “alkoxy” as used herein refers to an ether of the formula —O-alkyl. The term “C₁₋₁₀ alkoxy” as used herein refers to alkoxy wherein the alkyl is C₁₋₁₀ alkyl as described above. The term “polyalkylene oxide” refers to an ether comprising a repeating —(O-alkylene)- unit, e.g. —(OCH₂CH₂)ₙ—OH, or —(OCH₂CH₂)ₙ—OCH₃, wherein n is 1-4. In some embodiments, the ether attaching at an oxygen atom may be selected from the group consisting of: —O—R^{V}, —O—R^{W}—O—R^{X}, —O—R^{W}—O—R^{V}— O—R^{X}, or —O—R^{W}—O—R^{Y}—O—R^{Z}—O—R^{X}, wherein R^{V} is C₁₋₁₀ alkyl, R^{W} is C₂₋₁₀ alkyl, R^{Y} is C₂₋₈ alkyl, and R^{z} is C₂₋₆ alkyl, and R^{X} is H or C₂₋₈ alkyl, provided that the ether has from 1, 2, 3, 4, 5, 6, 7, 8, 9, or 10 carbon atoms.

The term “work function” has the ordinary meaning known to one of ordinary skill in the art. In some embodiments, the “work function” of a metal refers to a measure of the minimum energy required to extract an electron from the surface of the metal.

The term “high work function metal” has the ordinary meaning known to one of ordinary skill in the art. In some embodiments, a “high work function metal” is a metal or alloy that easily injects holes and typically has a work function greater than or equal to 4.5.

The term “low work function metal” has the ordinary meaning known to one of ordinary skill in the art. In some embodiments, a “low work function metal” is a metal or alloy that easily loses electrons and typically has a work function less than 4.3.

The term “deep blue emitting” has the ordinary meaning known to one of ordinary skill in the art. In some embodiments, a material is “deep blue emitting” if it emits deep blue light. Deep blue light is light having the approximate CIE color coordinates (X=[0.14], Y=[0.08], CIE 1931).

Some embodiments provide compounds that are useful as deep blue emitters. Formula 1 and Formula 2 represent examples of such compounds.

With respect to Formula 1 and Formula 2, X may be O or a single bond. Thus, some embodiments are related to compounds represented by one of Formulas 1a, 1b, 2a, and 2b.

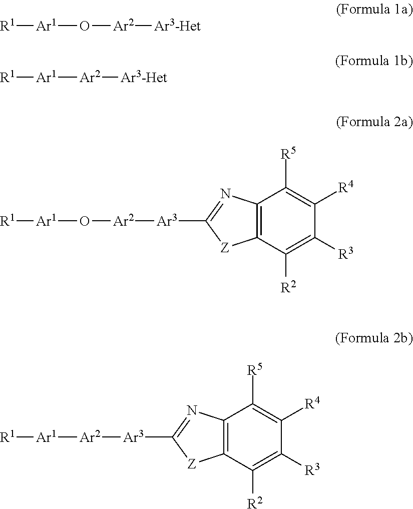

With respect to Formula 1, Formula 1a, Formula 1b, Formula 2, Formula 2a and Formula 2b, Ar³ may be optionally substituted aryl or Ar³ may be a single bond. Thus, some embodiments are related to compounds represented by Formulas Ic and 2c.

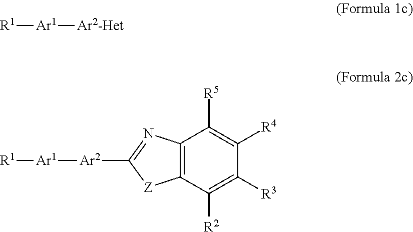

Some embodiments provide compounds represented by Formula 3 or 4:

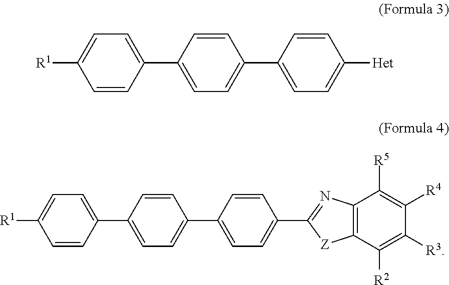

Some embodiments provide compounds represented by Formula 5, Formula 6, Formula 7, Formula 8, or Formula 9:

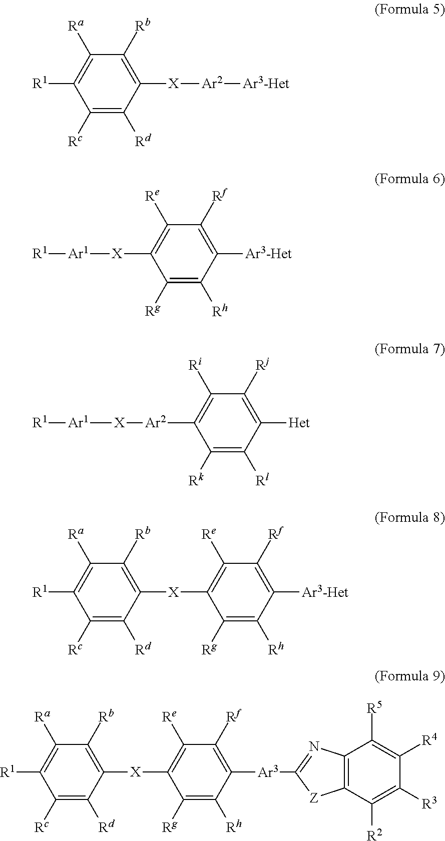

With respect to any relevant formula above, Het may be optionally substituted heteroaryl, such as optionally substituted C₆₋₁₀ heteroaryl, including, but not limited to optionally substituted benzooxazol-2-yl, optionally substituted benzothiazol-2-yl, optionally substituted benzoimidazol-2-yl, etc.

With respect to any relevant formula above, Ar¹ and Ar² may independently be optionally substituted aryl, and Ar³ may be optionally substituted aryl; or Ar³ may be a single bond. Ar¹, Ar², Ar³ (if present), and Het are independently optionally substituted. For example, in some embodiments, Ar¹ may be unsubstituted, or may have 1, 2, 3, or 4 substituents. In some embodiments, Ar² may be unsubstituted, or may have 1, 2, 3, or 4 substituents. In some embodiments, Ar³ may be unsubstituted, or may have 1, 2, 3, or 4 substituents. In some embodiments, Het may be unsubstituted, or may 1, 2, 3, or 4 substituents.

Some substituents of any of Ar¹, Ar², Ar³ (if present), and Het may include, but are not limited to, C₁₋₁₀ alkyl such as methyl, ethyl, propyl isomers (e.g. n-propyl and isopropyl), cyclopropyl, butyl isomers, cyclobutyl isomers (e.g. cyclobutyl, methylcyclopropyl, etc.), pentyl isomers, cyclopentyl isomers, hexyl isomers, cyclohexyl isomers, heptyl isomer, cycloheptyl isomers, etc; alkoxy such as —OCH₃, —OC₂H₅, —OC₃H₇, —OC₄H₉, —OC₅H₁₁, —OC₆H₁₃, —OC₇H₁₅, etc.; halo, such as F, Cl, Br, I, etc.; C₁₋₁₀ haloalkyl, including perfluoroalkyl such as —CF₃, —C₂F₅, —C₃F₇, —C₄F₉, etc.; C₁₋₁₀ acyl such as formyl, acetyl, benzoyl, etc.; C₁₋₁₀ amides attaching at the carbonyl or nitrogen atom such as —NCOCH₃, —CONHCH₂, etc.; C₁₋₁₀ esters attaching at the carbonyl or oxygen atom such as —OCOCH₃, —CO₂CH₂, etc.; C₁₋₁₀ carbamates attaching at the nitrogen atom or oxygen atom; cyano; cyanate; isocyanate; nitro; etc.

Also with respect to any relevant formula above, in some embodiments Het may comprise at least one electron-withdrawing substituent. In some embodiments, the electron-withdrawing substituent is a better electron withdrawer than a hydrogen atom. Examples include, but are not limited to, cyano, cyanate, isocyanate, nitro, F, Cl, perfluoralkyl, acyl, esters that attach at the carbonyl, or amides that attach at the carbonyl.

Also with respect to any relevant formula above, in some embodiments Ar¹ may comprise at least one electron-donating substituent. In some embodiments, the electron-donating substituent may be a better electron donor than a hydrogen atom. Examples include, but are not limited to alkyl, ethers attaching at an oxygen atom such as alkoxy, aryloxy or polyalkylene oxide, amino (e.g. —NR′R″, wherein R′ and R″ are independently H or alkyl), hydroxyl, etc.

Also with respect to any relevant formula above, R¹ is a C₁₋₁₀O₁₋₄ ether attaching at an oxygen atom, or —R⁷—NR⁸R⁹, wherein R⁷ is a single bond, optionally substituted C₆₋₁₀ aryloxy, or optionally substituted C₆₋₁₀ aryl, and R⁸ and R⁹ are independently C₆₋₁₀ aryl optionally substituted with one or more R¹⁰, wherein one of R¹⁰ on each of R⁸ and R⁹ optionally link together form a third ring comprising N. In some embodiments each of R¹, R⁷, R⁸, and R⁹ may independently be unsubstituted, or may have 1, 2, 3, 4, or 5 substituents. In some embodiments, the substituents of R¹, R⁷, R⁸, and R⁹ may be F, Cl, —R′, —OR′, or —NR′R″, wherein each R′ and R″ is independently H, optionally substituted phenyl, C₁₋₁₂ alkyl, or C₁₋₆ alkyl.

In some embodiments, R¹ may be optionally substituted C₁₂₋₃₀ diarylamino, such as optionally substituted diphenylamino, optionally substituted phenylnapthylenamino, optionally substituted phenylanthracenamino, etc.; optionally substituted carbazolyl; or a C₁₋₁₀O₁-4 ether attaching at an oxygen atom such as alkoxy (e.g. —OCH₃, —OC₂H₅, —OC₃H₇, —OC₄H₉, —OC₅H₁₁, —OC₆H₁₃, etc.), or polyalkylene oxide (e.g. —OCH₂CH₂OH, —OCH₂CH₂OCH₃, —(OCH₂CH₂)₂OH, —(OCH₂CH₂)₂OCH₃, —(OCH₂CH₂)₃OH, —(OCH₂CH₂)₃OCH₃, —(OCH₂CH₂)₄OH, —(OCH₂CH₂)₄OCH₃, etc). In some embodiments, R¹ is optionally substituted C₁₂₋₃₀ diarylamino, optionally substituted carbazolyl, optionally substituted C₁₈₋₃₆ diarylaminophenoxy, optionally substituted carbazolylphenoxy, or a C₁₋₁₀O₁₄ ether attaching at an oxygen atom. In some embodiments, R¹ is substituted C₁₂₋₃₀ diarylamino, substituted carbazolyl, optionally substituted C₁₈₋₃₆ diarylaminophenoxy, optionally substituted carbazolylphenoxy, or a C₁₋₁₀O₁₋₄ ether attaching at an oxygen atom. In some embodiments, R¹ may be optionally substituted carbazolyl, optionally substituted diphenyl amine, optionally substituted p-carbazolylphenoxy, optionally substituted p-diphenylaminophenoxy, or C₁₋₁₀ alkoxy. In some embodiments, any substituent of any of R¹, R⁷, R⁸, and R⁹ may be C₁₋₆ alkyl, C₁₋₆ alkoxy, optionally substituted phenyl, In some embodiments, R¹ may be methoxy,

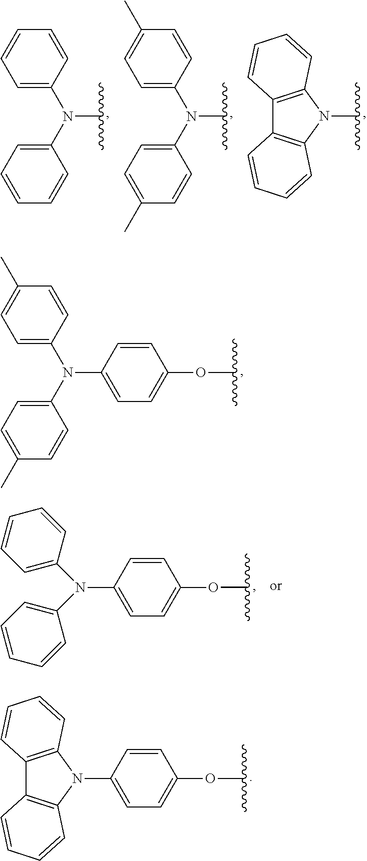

Also with respect to any relevant formula above, R², R³, R⁴, and R⁵ may independently be any substituents. In some embodiments, R², R³, R⁴, and R⁵ may be independently H, optionally substituted C₆₋₃₀ aryl; such as optionally substituted phenyl, C₁₋₁₀ alkyl, such as methyl, ethyl, propyl isomers (e.g. n-propyl and isopropyl), cyclopropyl, butyl isomers, cyclobutyl isomers (e.g. cyclobutyl, methylcyclopropyl, etc.), pentyl isomers, cyclopentyl isomers, hexyl isomers, cyclohexyl isomers, heptyl isomer, cycloheptyl isomers, etc or C₁₋₁₀ alkoxy, alkoxy such as —OCH₃, —OC₂H₅, —OC₃H₇, —OC₄H₉, —OC₅H₁₁, —OC₆H₁₃, —OC₇H₁₅, etc.

Also with respect to any relevant formula above, R^{a}, R^{b}, R^{c}, R^{d}, R^{e}, R^{f}, R^{g}, R^{h}, Rⁱ, R^{k}, and R^{l} may be any substituent. In some embodiments, R^{a}, R^{b}, R^{c}, R^{d}, R^{e}, R^{f}, R^{g}, R^{h}, Rⁱ, R^{j}, R^{k}, and R^{l} may be independently selected from C₁₋₁₀ alkyl and halo. In some embodiments, R^{a}, R^{b}, R^{c}, R^{d}, R^{e}, R^{f}, R^{g}, R^{h}, Rⁱ, R^{j}, R^{k}, and R^{l} may be independently selected from C₁₋₃ alkyl, F, and Cl.

Also with respect to any relevant formula above, in some embodiments at least one of Ar¹, Ar², and Ar³ (if present) may be optionally substituted p-interphenylene. In some embodiments, each of Ar¹, Ar², and Ar³ (if present) may independently be optionally substituted p-interphenylene. In some embodiments, Ar¹, Ar², and Ar³ (if present) may independently have 0, 1, or 2 substituents independently selected from C₁₋₃ alkyl, F, and Cl. In some embodiments, at least one of Ar¹, Ar², and Ar³ (if present) may be unsubstituted p-interphenylene. In some embodiments, each of Ar¹, Ar², and Ar³ (if present) may be unsubstituted p-interphenylene.

Also with respect to any relevant formula above, in some embodiments Z may be O, S, or NR⁶ wherein R⁶ is optionally substituted phenyl. In some of embodiments, Z may be O, S, or NR⁶ wherein R⁶ is optionally substituted phenyl; and R¹ may be optionally substituted diphenyl amine, optionally substituted carbazolyl, optionally substituted p-carbazolylphenoxy, or optionally substituted p-diphenylaminophenoxy.

Also with respect to any relevant formula above, in some embodiments R¹ is optionally substituted diphenyl amine or optionally substituted carbazolyl.

Also with respect to any relevant formula above, in some embodiments Ar³ is aryl having 0, 1, or 2 substituents independently selected from C₁₋₃ alkyl, F, and Cl. In some embodiments, Ar³ is aryl having 0, 1, or 2 substituents independently selected from C₁₋₃ alkyl, F, and Cl; and R¹ is optionally substituted diphenyl amine, or optionally substituted carbazolyl.

Also with respect to any relevant formula above, in some embodiments —Ar¹—X—Ar²—Ar³— is not

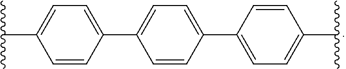

Some embodiments relate to optionally substituted Ring Systems 1-9.

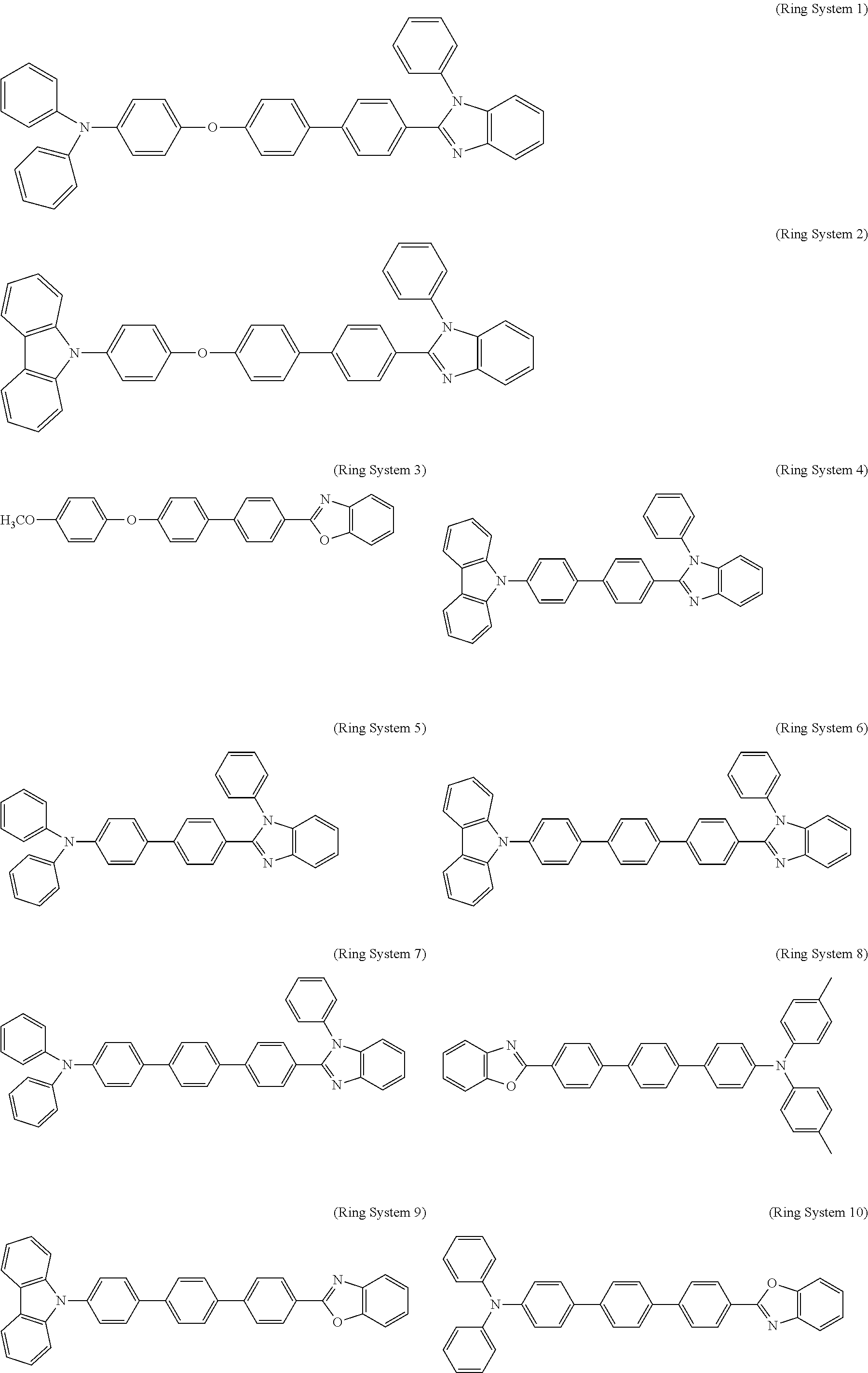

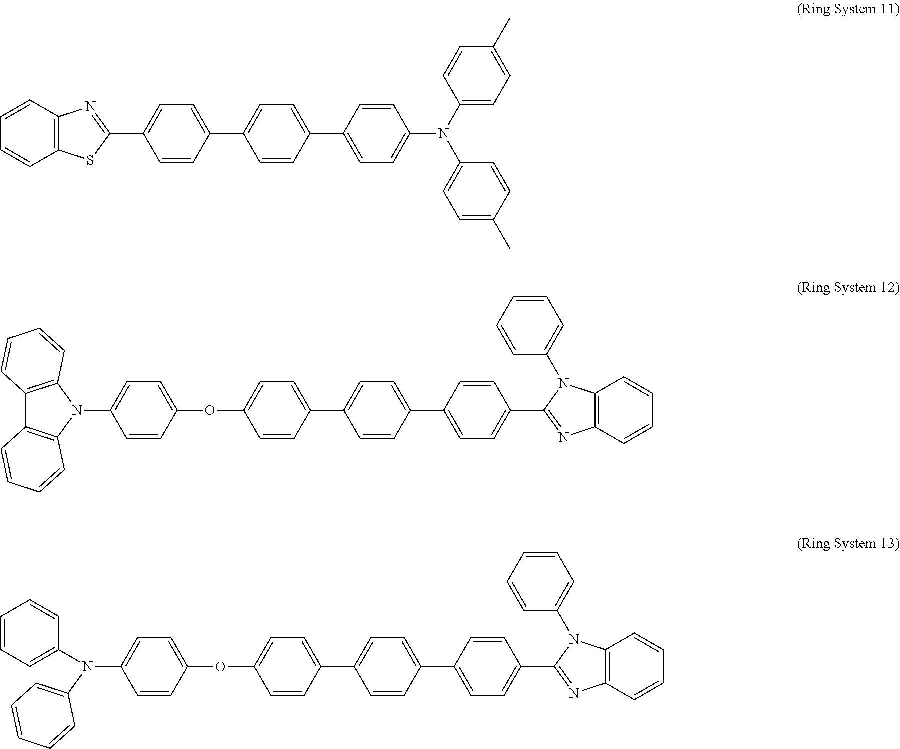

In these embodiments, the ring systems may have any substituent described above, including those described with respect to Ar¹, Ar², Ar³, and Het. In some embodiments, Ring Systems 1-7 may have 0, 1, 2, 3, 4, 5, or 6 substituents. In some embodiments, the substituents are independently selected from: C₁₋₆ alkyl, C₁₋₆ alkoxy, F, Cl, Br, and I.

Some embodiments relate to a compound selected from:

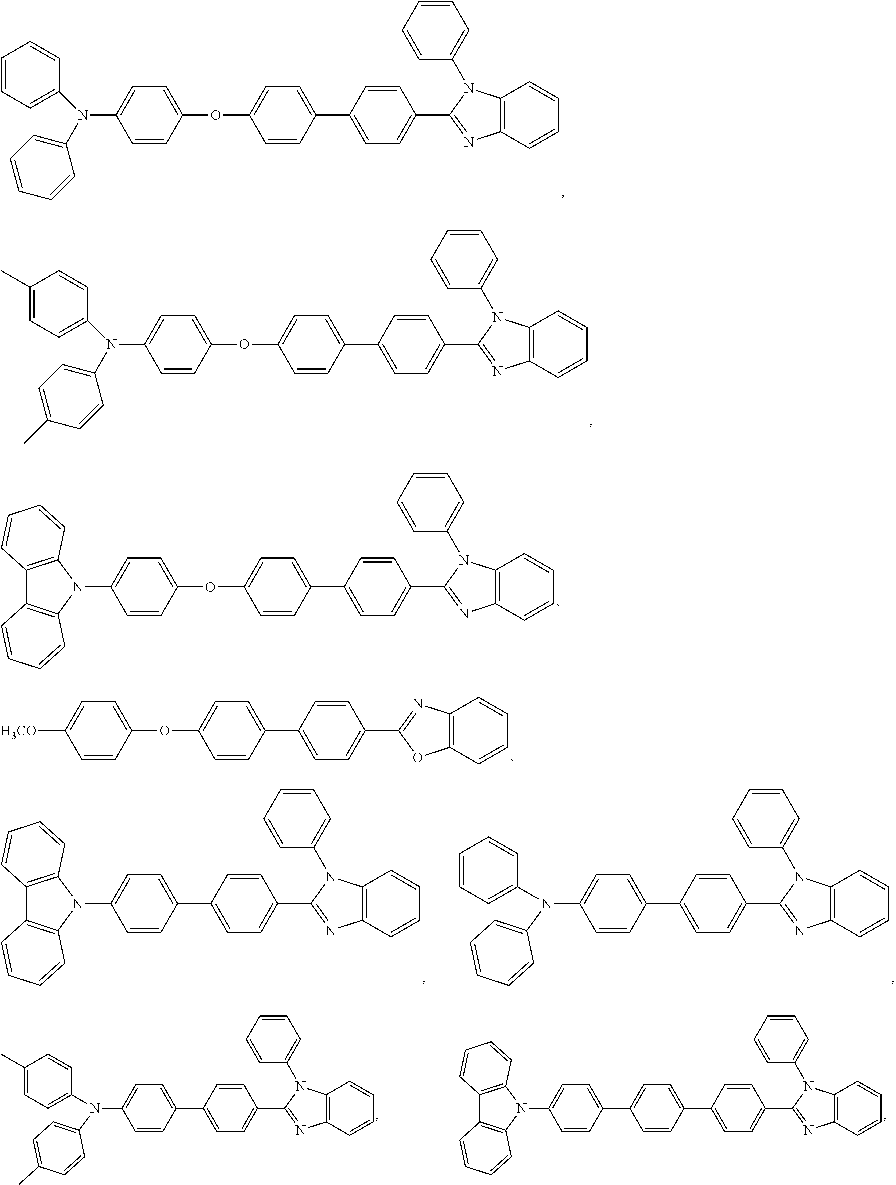

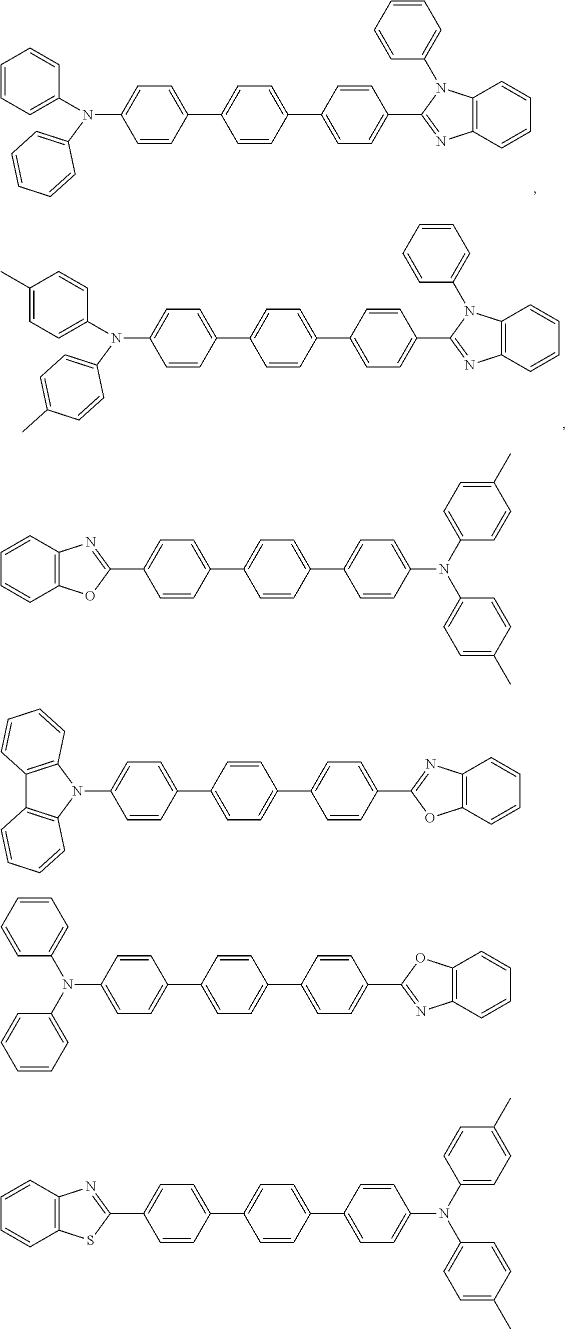

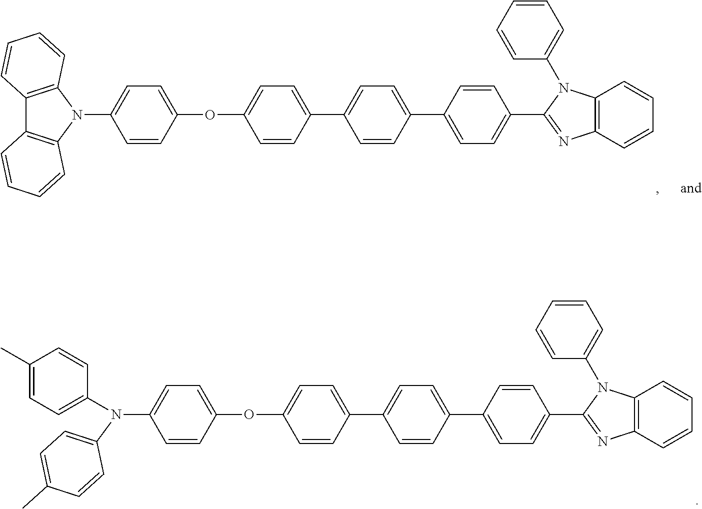

The compounds and compositions described herein can be incorporated into light-emitting devices in various ways. For example, an embodiment provides a light-emitting device comprising: an anode layer (e.g., an anode layer comprising a high work function metal); a cathode layer (e.g., a cathode layer comprising a low work function metal); and a light-emitting layer positioned the anode layer and the cathode layer. In some embodiments, the device is configured so that electrons can be transferred from the cathode to the light-emitting layer and holes can be transferred from the anode to the light-emitting layer. The light-emitting layer comprises the compounds and/or compositions disclosed herein.

An anode layer may comprise a conventional material such as a metal, mixed metal, alloy, metal oxide or mixed-metal oxide, or a conductive polymer. Examples of suitable metals include the metals in Groups 10, Group 11, and Group 12 transition metals. If the anode layer is to be light-transmitting, mixed-metal oxides of Groups 12, Group 13, and Group 14 metals or alloys thereof, such as zinc oxide, tin oxide, indium zinc oxide (IZO) or indium-tin-oxide (ITO) may be used. The anode layer may include an organic material such as polyaniline, e.g., as described in “Flexible light-emitting diodes made from soluble conducting polymer,” Nature, vol. 357, pp. 477-479 (11 Jun. 1992). Examples of suitable high work function metals include but are not limited to Au, Pt, indium-tin-oxide (ITO), or alloys thereof. In some embodiments, the anode layer can have a thickness in the range of about 1 nm to about 1000 nm.

A cathode layer may include a material having a lower work function than the anode layer. Examples of suitable materials for the cathode layer include those selected from alkali metals of Group 1, Group 2 metals, Group 11, Group 12, and Group 13 metals including rare earth elements, lanthanides and actinides, materials such as aluminum, indium, calcium, barium, samarium and magnesium, and combinations thereof. Li-containing organometallic compounds, LiF, and Li₂O may also be deposited between the organic layer and the cathode layer to lower the operating voltage. Suitable low work function metals include but are not limited to Al, Ag, Mg, Ca, Cu, Mg/Ag, LiF/Al, CsF, CsF/Al or alloys thereof. In some embodiments, the cathode layer can have a thickness in the range of about 1 nm to about 1000 nm.

The amount of the compounds disclosed herein in the light-emitting composition can vary. In some embodiments, the light-emitting layer consists essentially of a compound disclosed herein. In other embodiments, the emissive layer comprises a host material and at least one of the emissive compounds disclosed herein. If there is a host material, the amount of the emissive compound with respect to the host material may be any amount suitable to produce adequate emission. In some embodiments, the amount of a compound disclosed herein in the light-emitting layer is in the range of from about 1% to about 100% by weight of the light-emitting layer. In embodiments where a compound disclosed herein is used as a host, the compound may be about 80% or about 90% to about 99% by weight, of the light-emitting layer. In embodiments where a compound disclosed herein is used as an emissive compound, the compound may be about 1% to about 10%, or alternatively, about 3% by weight of the light-emitting layer.

The thickness of the light-emitting layer may vary. In some embodiments, the light-emitting layer has a thickness in the range of from about 20 nm to about 150 nm, or from about 20 nm to about 200 nm.

The host in the emissive layer may be at least one of: one or more hole-transport materials, one or more electron-transport materials, and one or more ambipolar materials, which are materials understood by those skilled in the art to be capable of transporting both holes and electrons.

In some embodiments, the hole-transport material comprises at least one of an aromatic-substituted amine, a carbazole, a polyvinylcarbazole (PVK), e.g. poly(9-vinylcarbazole); N,N′-bis(3-methylphenyl)N,N′-diphenyl-[1,1′-biphenyl]-4,4′-diamine (TPD); polyfluorene; a polyfluorene copolymer; poly(9,9-di-n-octylfluorene-alt-benzothiadiazole); poly(paraphenylene); poly[2-(5-cyano-5-methylhexyloxy)-1,4-phenylene]; 1,1-Bis(4-bis(4-methylphenyl)aminophenyl)cyclohexane; 2,9-Dimethyl-4,7-diphenyl-1,10-phenanthroline; 3,5-Bis(4-tert-butyl-phenyl)-4-phenyl[1,2,4]triazole; 3,4,5-Triphenyl-1,2,3-triazole; 4,4′,4″-Tris(N-(naphthylen-2-yl)-N-phenylamino)triphenylamine; 4,4′,4′-tris(3-methylphenylphenylamino)triphenylamine (MTDATA); 4,4′-bis[N-(naphthyl)-N-phenyl-amino]biphenyl (α-NPD); 4,4′-bis[N,N′-(3-tolyl)amino]-3,3′-dimethylbiphenyl (HMTPD); 4,4′-N,N′-dicarbazole-biphenyl (CBP); 1,3-N,N-dicarbazole-benzene (mCP); poly(9-vinylcarbazole) (PVK); a benzidine; a phenylenediamine; a phthalocyanine metal complex; a polyacetylene; a polythiophene; a triphenylamine; an oxadiazole; copper phthalocyanine; N,N′N″-1,3,5-tricarbazoloylbenzene (tCP); N,N′-bis(4-butylphenyl)-N,N′-bis(phenyl)benzidine; mixtures thereof, and the like.

In some embodiments, the electron-transport material comprises at least one of 2-(4-biphenylyl)-5-(4-tert-butylphenyl)-1,3,4-oxadiazole (PBD); 1,3-bis(N,N-t-butyl-phenyl)-1,3,4-oxadiazole (OXD-7), 1,3-bis[2-(2,2′-bipyridine-6-yl)-1,3,4-oxadiazo-5-yl]benzene; 3-phenyl-4-(1′-naphthyl)-5-phenyl-1,2,4-triazole (TAZ); 2,9-dimethyl-4,7-diphenyl-phenanthroline (bathocuproine or BCP); aluminum tris(8-hydroxyquinolate) (Alq3); and 1,3,5-tris(2-N-phenylbenzimidazolyl)benzene; 1,3-bis[2-(2,2′-bipyridine-6-yl)-1,3,4-oxadiazo-5-yl]benzene (BPY-OXD); 3-phenyl-4-(1′-naphthyl)-5-phenyl-1,2,4-triazole (TAZ), 2,9-dimethyl-4,7-diphenyl-phenanthroline (bathocuproine or BCP); and 1,3,5-tris[2-N-phenylbenzimidazol-z-yl]benzene (TPBI). In some embodiments, the electron transport layer is aluminum quinolate (Alq₃), 2-(4-biphenylyl)-5-(4-tert-butylphenyl)-1,3,4-oxadiazole (PBD), phenanthroline, quinoxaline, 1,3,5-tris[N-phenylbenzimidazol-z-yl]benzene (TPBI), or a derivative or a mixture thereof.

In some embodiments, the device comprises no electron transport or hole transport layer. In some embodiments, the device consists essentially of the anode layer, the cathode layer, and the light-emitting layer. In other embodiments, the light-emitting device may further comprise a hole-transport layer disposed between the anode and the light-emitting layer. The hole-transport layer may comprise at least one hole-transport material. Suitable hole-transport materials may include those listed above in addition to any others known to those skilled in the art. In some embodiments, the light-emitting device may further comprise an electron-transport layer disposed between the cathode and the light-emitting layer. The electron-transport layer may comprise at least one electron-transport material. Suitable electron transport materials include those listed above and any others known to those skilled in the art.

If desired, additional layers may be included in the light-emitting device. These additional layers may include an electron injection layer (EIL), a hole blocking layer (HBL), an exciton blocking layer (EBL), and/or a hole injection layer (HIL). In addition to separate layers, some of these materials may be combined into a single layer.

In some embodiments, the light-emitting device can include an electron injection layer between the cathode layer and the light emitting layer. A number of suitable electron injection materials are known to those skilled in the art. Examples of suitable material(s) that can be included in the electron injection layer include but are not limited to, an optionally substituted compound selected from the following: aluminum quinolate (Alq₃), 2-(4-biphenylyl)-5-(4-tert-butylphenyl)-1,3,4-oxadiazole (PBD), phenanthroline, quinoxaline, 1,3,5-tris[N-phenylbenzimidazol-z-yl]benzene (TPBI) a triazine, a metal chelate of 8-hydroxyquinoline such as tris(8-hydroxyquinoliate) aluminum, and a metal thioxinoid compound such as bis(8-quinolinethiolato) zinc. In some embodiments, the electron injection layer is aluminum quinolate (Alq₃), 2-(4-biphenylyl)-5-(4-tert-butylphenyl)-1,3,4-oxadiazole (PBD), phenanthroline, quinoxaline, 1,3,5-tris[N-phenylbenzimidazol-z-yl]benzene (TPBI), or a derivative or a combination thereof.

In some embodiments, the device can include a hole blocking layer, e.g., between the cathode and the light-emitting layer. Various suitable hole blocking materials that can be included in the hole blocking layer are known to those skilled in the art. Suitable hole blocking material(s) include but are not limited to, an optionally substituted compound selected from the following: bathocuproine (BCP), 3,4,5-triphenyl-1,2,4-triazole, 3,5-bis(4-tert-butyl-phenyl)-4-phenyl-[1,2,4]triazole, 2,9-dimethyl-4,7-diphenyl-1,10-phenanthroline, and 1,1-bis(4-bis(4-methylphenyl)aminophenyl)-cyclohexane.

In some embodiments, the light-emitting device can include an exciton blocking layer, e.g., between the light-emitting layer and the anode. In an embodiment, the band gap of the material(s) that comprise exciton blocking layer is large enough to substantially prevent the diffusion of excitons. A number of suitable exciton blocking materials that can be included in the exciton blocking layer are known to those skilled in the art. Examples of material(s) that can compose an exciton blocking layer include an optionally substituted compound selected from the following: aluminum quinolate (Alq₃), 4,4′-bis[N-(naphthyl)-N-phenyl-amino]biphenyl (α-NPD), 4,4′-N,N′-dicarbazole-biphenyl (CBP), and bathocuproine (BCP), and any other material(s) that have a large enough band gap to substantially prevent the diffusion of excitons.

In some embodiments, the light-emitting device can include a hole injection layer, e.g., between the light-emitting layer and the anode. Various suitable hole injection materials that can be included in the hole injection layer are known to those skilled in the art. Exemplary hole injection material(s) include an optionally substituted compound selected from the following: a polythiophene derivative such as poly(3,4-ethylenedioxythiophene (PEDOT)/polystyrene sulphonic acid (PSS), a benzidine derivative such as N,N,N′,N′-tetraphenylbenzidine, poly(N,N′-bis(4-butylphenyl)-N,N′-bis(phenyl)benzidine), a triphenylamine or phenylenediamine derivative such as N,N′-bis(4-methylphenyl)-N,N′-bis(phenyl)-1,4-phenylenediamine, 4,4′,4″-tris(N-(naphthylen-2-yl)-N-phenylamino)triphenylamine, an oxadiazole derivative such as 1,3-bis(5-(4-diphenylamino)phenyl-1,3,4-oxadiazol-2-yl)benzene, a polyacetylene derivative such as poly(1,2-bis-benzylthio-acetylene), and a phthalocyanine metal complex derivative such as phthalocyanine copper. Hole-injection materials, while still being able to transport holes, may have a hole mobility substantially less than the hole mobility of conventional hole transport materials.

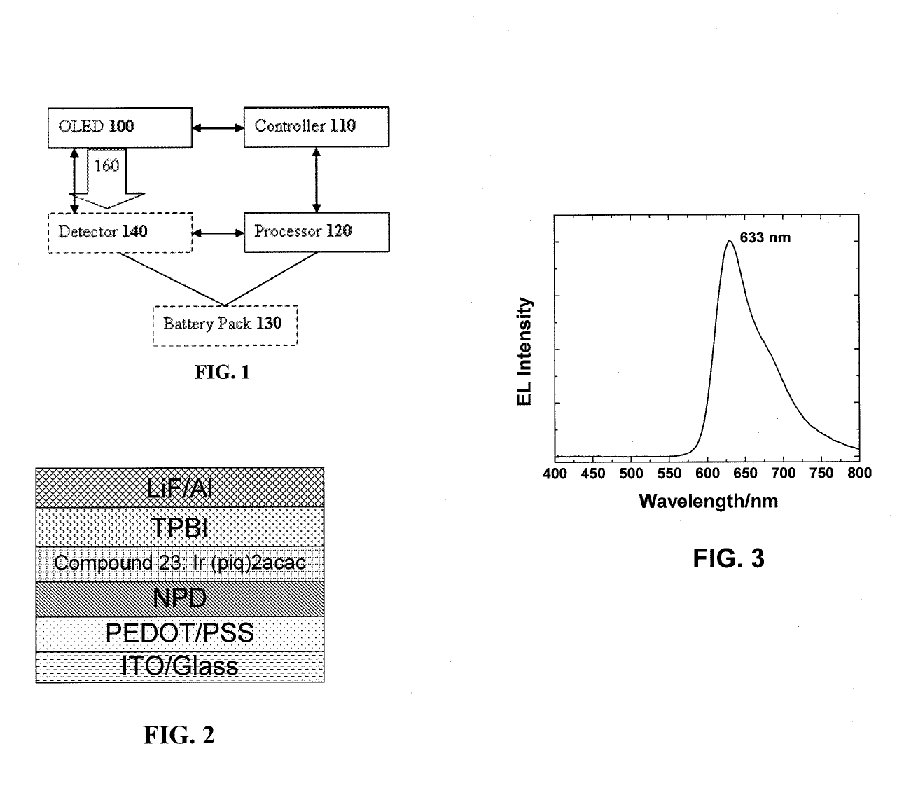

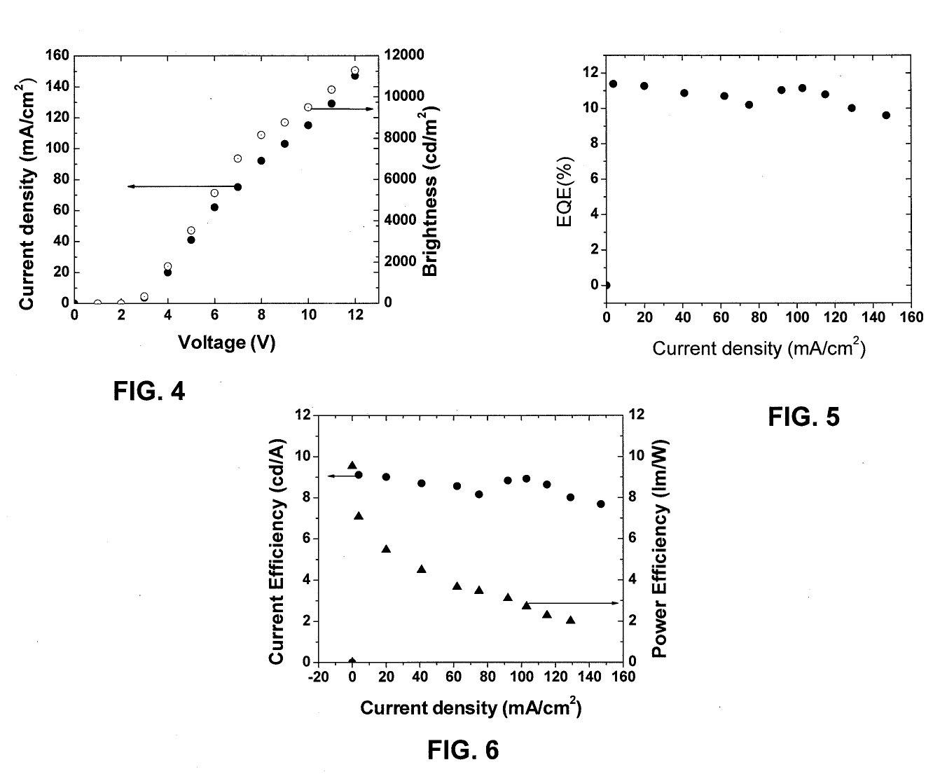

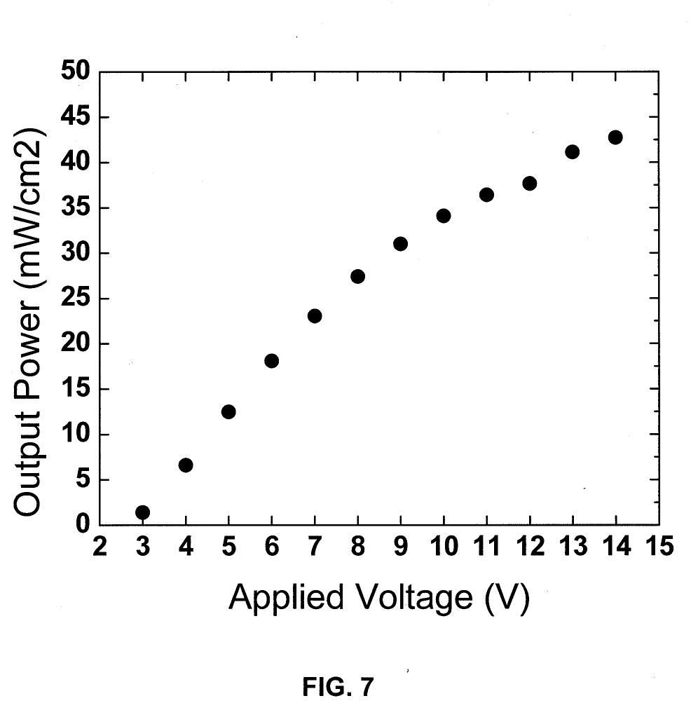

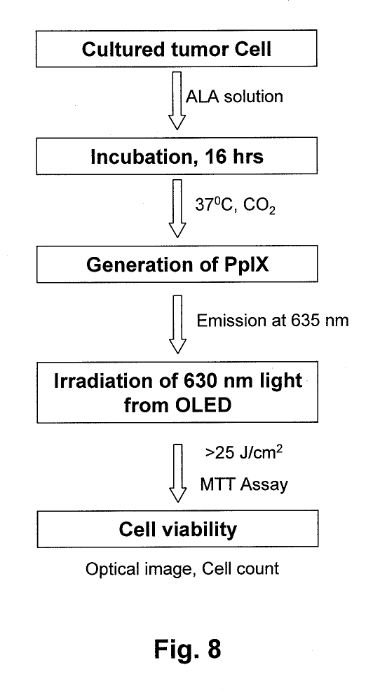

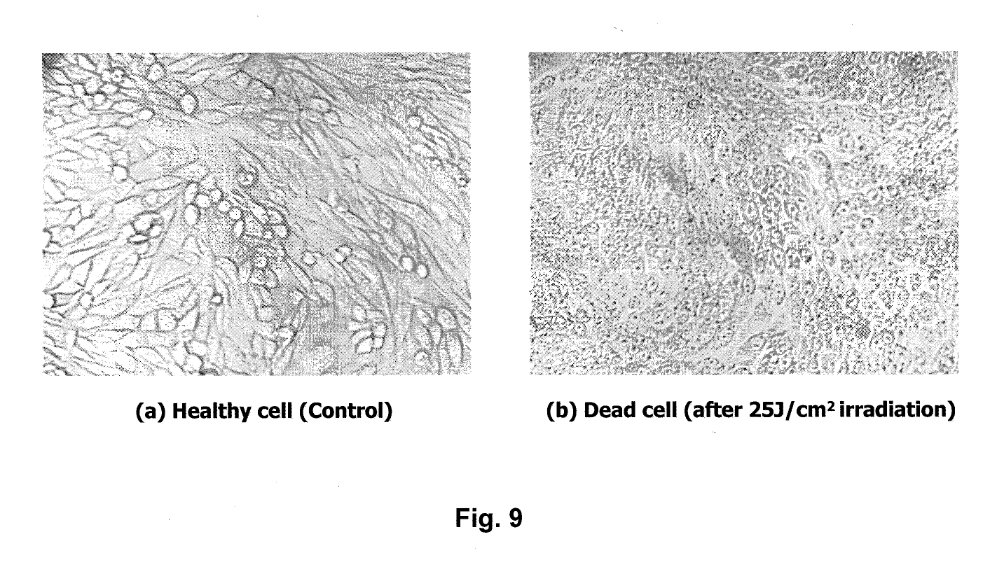

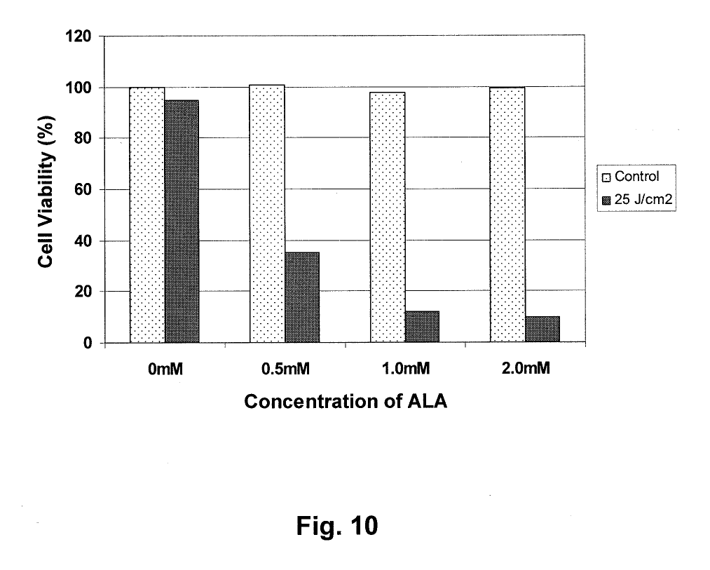

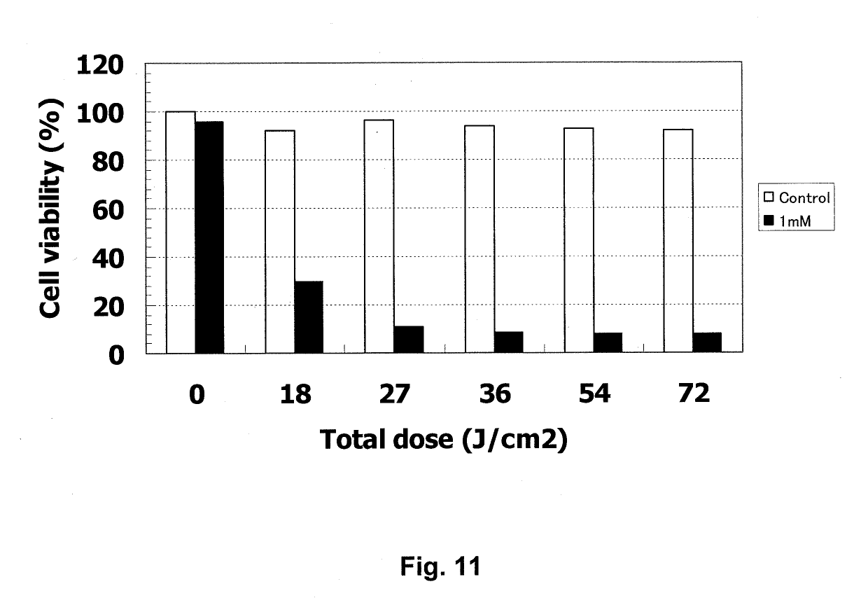

Those skilled in the art recognize that the various materials described above can be incorporated in several different layers depending on the configuration of the device. In one embodiment, the materials used in each layer are selected to result in the recombination of the holes and electrons in the light-emitting layer. An example of a device configuration that incorporates the various layers described herein is illustrated schematically in FIG. 1. The electron injection layer (EIL), electron transport layer (ETL), hole blocking layer (HBL), exciton blocking layer (EBL), hole transport layer (HTL), and hole injection layer (HIL) can be incorporated in the light-emitting device using methods known to those skilled in the art (e.g., vapor deposition).

The emissive compositions may be prepared by adapting methods known in the art for other emissive compositions. For example, the emissive compositions may be prepared by dissolving or dispersing the emissive compound in a solvent and depositing the compound on the appropriate layer of the device. The liquid may be a single phase, or may comprise one or more additional solid or liquid phases dispersed within the liquid. The solvent may then be allowed to evaporate, or the solvent may be removed via heat or vacuum, to provide an emissive composition. If a host is present, it may be dissolved or dispersed in the solvent with the emissive device and treated as explained above. Alternatively, the compound may be added to a molten or liquid host material, which is then allowed to solidify to provide a viscous liquid or solid emissive composition.

Light-emitting devices comprising the compounds disclosed herein can be fabricated using techniques known in the art, as informed by the guidance provided herein. For example, a glass substrate can be coated with a high work functioning metal such as ITO which can act as an anode. After patterning the anode layer, a light-emitting layer that includes at least a compound disclosed herein can be deposited on the anode. The cathode layer, comprising a low work functioning metal (e.g., Mg:Ag), can then be deposited, e.g., vapor evaporated, onto the light-emitting layer. If desired, the device can also include an electron transport/injection layer, a hole blocking layer, a hole injection layer, an exciton blocking layer and/or a second light-emitting layer that can be added to the device using techniques known in the art, as informed by the guidance provided herein.

In some embodiments, the light-emitting device (e.g., OLED) is configured by a wet process such as a process that comprises at least one of spraying, spin coating, drop casting, inkjet printing, screen printing, etc. Some embodiments provide a composition which is a liquid suitable for deposition onto a substrate. The liquid may be a single phase, or may comprise one or more additional solid or liquid phases dispersed in it. The liquid typically comprises a light-emitting compound, a host material disclosed herein and a solvent.

### Phototherapy

The devices disclosed herein may be useful in phototherapy. Typically, phototherapy involves exposing at least a portion of the tissue of a mammal with light, such as light from a device described herein.

The phototherapy may have a therapeutic effect, such as the diagnosis, cure, mitigation, treatment, or prevention of disease, or otherwise affecting the structure or function of the body of man or other animals. Some examples of conditions that phototherapy may be useful to treat or diagnose include, but are not limited to, infection, cancer/tumors, cardiovascular conditions, dermatological conditions, a condition affecting the eye, obesity, pain or inflammation, conditions related to immune response, etc.

Examples of infections may include microbial infection such as bacterial infection, viral infection, fungus infection, protozoa infection, etc.

Exemplary cancer or tumor tissues include vascular endothelial tissue, an abnormal vascular wall of a tumor, a solid tumor, a tumor of a head, a tumor of the brain, a tumor of a neck, a tumor of a gastrointestinal tract, a tumor of a liver, a tumor of a breast, a tumor of a prostate, a tumor of a lung, a nonsolid tumor, malignant cells of one of a hematopoietic tissue and a lymphoid tissue, lesions in a vascular system, a diseased bone marrow, diseased cells in which the disease is one of an autoimmune and an inflammatory disease, etc.

Examples of cardiovascular conditions may include myocardial infarction, stroke, lesions in a vascular system, such as atherosclerotic lesions, arteriovenous malformations, aneurysms, venous lesions, etc. For example, a target vascular tissue may be destroyed by cutting off circulation to the desired location.

Examples of dermatological conditions may include hair loss, hair growth, acne, psoriasis, wrinkles, discoloration, skin cancer, rosacea, etc.

Examples of eye conditions may include age related macular degeneration (AMD), glaucoma, diabetic retinopathy, neovascular disease, pathological myopia, ocular histoplasmosis, etc.

Examples of pain or inflammation include arthritis, carpal tunnel, metatarsalgia, plantar fasciitis, TMJ, pain or inflammation affecting an elbow, an ankle, a hip, a hand, etc. Examples of conditions related to immune response include, HIV or other autoimmune disease, organ transplant rejection, etc.

Other non-limiting uses of phototherapy may include treating benign prostate hyperplasia, treating conditions affecting adipose tissue, wound healing, inhibiting cell growth, and preserving donated blood.

The light itself may be at least partially responsible for the therapeutic effects of the phototherapy, thus phototherapy may be carried out without a photosensitive compound. In embodiments where a photosensitive compound is not used, light in the red range (approximately 630 nm to 700 nm) may decrease inflammation in injured tissue, increase ATP production, and otherwise stimulate beneficial cellular activity. Light in the red range may also be used in conjunction with light of other spectral wavelengths, for example blue or yellow, to facilitate post operative healing. Facial rejuvenation may be effected by applying about 633 nm radiation to the desired tissue for about 20 minutes. In some embodiments, facial skin rejuvenation is believed to be attained by applying light in the red range for a therapeutically effective amount of time.

The light may also be used in conjunction with a photosensitive compound. The photosensitive compound may be administered directly or indirectly to body tissue so that the photosensitive compound is in or on the tissue. At least a portion of the photosensitive compound may then be activated by exposing at least a portion of tissue with light.

For example, a photosensitive compound may be administered systemically by ingestion or injection, topically applying the compound to a specific treatment site on a patient's body, or by some other method. This may be followed by illumination of the treatment site with light having a wavelength or waveband corresponding to a characteristic absorption waveband of the photosensitive compound, such as about 500 or about 600 nm to about 800 nm or about 1100 nm, which activates the photosensitive compound. Activating the photosensitive compound may cause singlet oxygen radicals and other reactive species to be generated, leading to a number of biological effects that may destroy the tissue which has absorbed the photosensitive compound such as abnormal or diseased tissue.

The photosensitive compound may be any compound or pharmaceutically acceptable salts or hydrates thereof, which react as a direct or indirect result of absorption of ultraviolet, visible, or infrared light. In one embodiment, the photosensitive compound reacts as a direct or indirect result of absorption of red light. The photosensitive compound may be a compound which is not naturally in the tissue. Alternatively, the photosensitive compound may naturally be present in the tissue, but an additional amount of the photosensitive compound may be administered to the mammal. In some embodiments, the photosensitive compound may selectively bind to one or more types of selected target cells and, when exposed to light of an appropriate waveband, absorb the light, causing substances to be produced that impair or destroy the target cells.

While not limiting any embodiment, for some types of therapies, it may be helpful if the photosensitive compound has low enough toxicity so as not to cause more harm than the disease or the condition that is to be treated with the phototherapy to which it is administered or is capable of being formulated in a composition with sufficiently low toxicity that can be administered to the animal. In some embodiments, it may also be helpful if the photodegradation products of the photosensitive compounds are nontoxic.

Some non-limiting examples of photosensitive chemicals may be found in Kreimer-Bimbaum, Sem. Hematol, 26:157-73, (1989), incorporated by reference herein in its entirety, and include, but are not limited to, chlorins, e.g., Tetrahydroxylphenyl chlorin (THPC) [652 nm], bacteriochlorins [765 nm], e.g., N-Aspartyl chlorin e6 [664 nm], phthalocyanines [600-700 nm], porphyrins, e.g., hematoporphyrin [HPD][630 nm], purpurins, e.g., [1,2,4-Trihydroxyanthraquinone] Tin Etiopurpurin [660 nm], merocyanines, psoralens, benzoporphyrin derivatives (BPD), e.g., verteporfin, and porfimer sodium; and pro-drugs such as delta-aminolevulinic acid or methyl aminolevulinate, which can produce photosensitive agents such as protoporphyrin IX. Other suitable photosensitive compounds include indocyanine green (ICG) [800 nm], methylene blue [668 nm, 609 nm], toluidine blue, texaphyrins, Talaportin Sodium (mono-L-aspartyl chlorine)[664 nm], verteprofin [693 nm], which may be useful for phototherapy treatment of conditions such as age-related macular degeneration, ocular histoplasmosis, or pathologic myopia], lutetium texaphyrin [732 nm], and rostaporfin [664 nm].

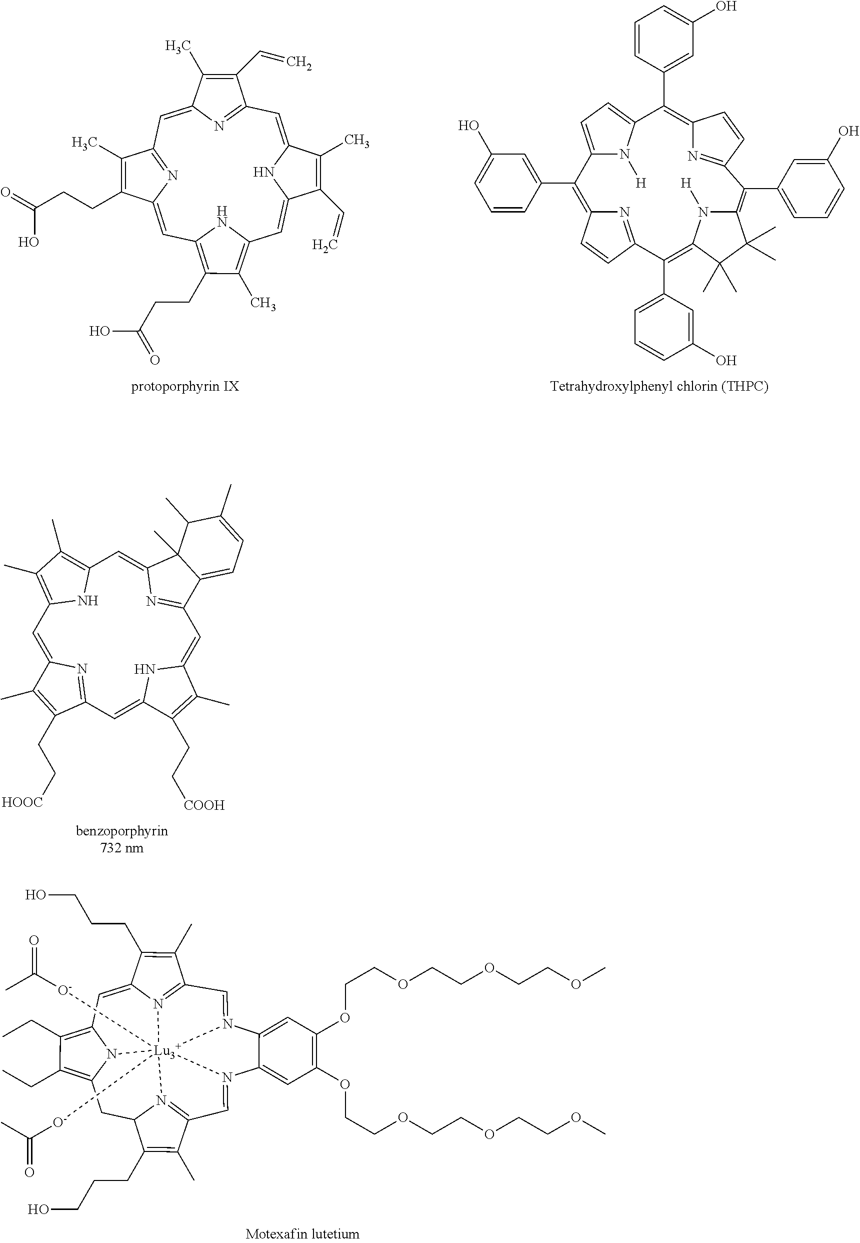

In some embodiments, the photosensitive compound comprises at least one component of porfimer sodium. Porfimer sodium comprises a mixture of oligomers formed by ether and ester linkages of up to eight porphorin units. The structural formula below is representative of some of the compounds present in porfimer, wherein n is 0, 1, 2, 3, 4, 5, or 6 and each R is independently —CH(OH)CH₃ or —CH═CH₂.

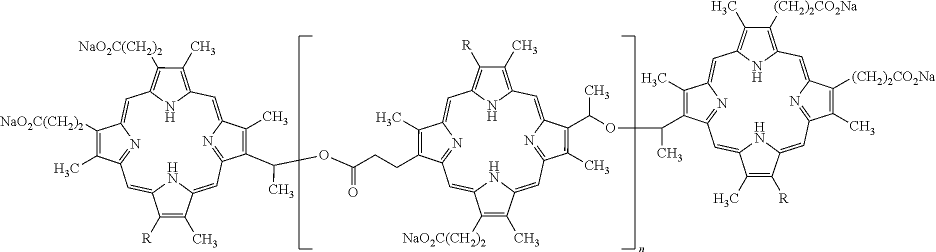

In some embodiments, the photosensitive compound is at least one of the regioisomers of verteporphin, shown below.

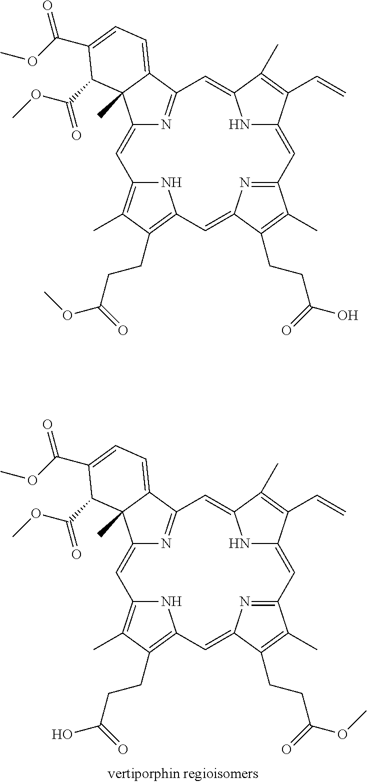

In some embodiments, the photosensitive compound comprises a metal analogue of phthalocyanine shown below.

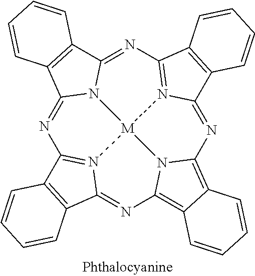

In one embodiment, M is zinc. In one embodiment, the compound can be zinc phthalocyanine or zinc phthalocyanine tetrasulfonate.

A photosensitive agent can be administered in a dry formulation, such as a pill, a capsule, a suppository or a patch. The photosensitive agent may also be administered in a liquid formulation, either alone, with water, or with pharmaceutically acceptable excipients, such as those disclosed in Remington's Pharmaceutical Sciences. The liquid formulation also can be a suspension or an emulsion. Liposomal or lipophilic formulations may be desirable. If suspensions or emulsions are utilized, suitable excipients may include water, saline, dextrose, glycerol, and the like. These compositions may contain minor amounts of nontoxic auxiliary substances such as wetting or emulsifying agents, antioxidants, pH buffering agents, and the like. The above described formulations may be administered by methods which may include but are not limited to intradermal, intramuscular, intraperitoneal, intravenous, subcutaneous, intranasal, epidural, oral, sublingual, intranasal, intracerebral, intravaginal, transdermal, iontophoretical, rectally, by inhalation, or topically to the desired target area, for example, the body cavity (oral, nasal, rectal), ears, nose, eyes, or skin. The preferred mode of administration is left to the discretion of the practitioner, and will depend in-part upon the site of the medical condition (such as the site of cancer or viral infection).

The dose of photosensitive agent may vary. For example, the target tissue, cells, or composition, the optimal blood level, the animal's weight, and the timing and duration of the radiation administered, may affect the amount of photosensitive agent used. Depending on the photosensitive agent used, an equivalent optimal therapeutic level may have to be empirically established. The dose may be calculated to obtain a desired blood level of the photosensitive agent, which in some embodiments may be from about 0.001 g/mL or 0.01 μg/ml to about 100 μg/ml or about 1000 μg/ml.

In some embodiments, about 0.05 mg/kg or about 1 mg/kg to about 50 mg/kg or about 100 mg/kg is administered to the mammal. Alternatively, for topical application, about 0.15 mg/m² or about 5 mg/m² to about 30 mg/m² or about 50 mg/m² may be administered to the surface of the tissue.

The light may be administered by an external or an internal light source, such as an OLED device described herein. The intensity of radiation or light used to treat the target cell or target tissue may vary. In some embodiments, the intensity may be about 0.1 mW/cm² to about 100 mW/cm², about 1 mW/cm² to about 50 mW/cm², or about 3 mW/cm² to about 30 mW/cm². The duration of radiation or light exposure administered to a subject may vary. In some embodiments the exposure ranges from about 1 minute, about 60 minutes, or about 2 hours to about 24 hours, about 48 hours, or about 72 hours.

A certain amount of light energy may be required to provide a therapeutic effect. For example, a certain amount of light energy may be required to activate the photosensitive compounds. This may be accomplished by using a higher power light source, which may provide the needed energy in a shorter period of time, or a lower power light source may be used for a longer period of time. Thus, a longer exposure to the light may allow a lower power light source to be used, while a higher power light source may allow the treatment to be done in a shorter time. In some embodiments, the total fluence or light energy administered during a treatment may be in the range of 5 Joules to 1,000 Joules, 20 Joules to 750 Joules, or 50 Joules to 500 Joules.

FIG. 1 is a schematic of some embodiments which further include a controller 110 and processor 120 electrically connected to an organic light-emitting diode 100 (OLED), which may help to provide a uniform power supply to facilitate homogeneous light exposure of the tissue. In some embodiments, the apparatus further includes an optional detector 140, such as photodiode, which detects a portion of the light 160 emitted from the OLED 100, to help determine the amount of light being emitted by the OLED 100. For example, the detector 140 may communicate a signal related to the intensity of the light 160 received from the OLED 100 to the processor 120, which, based upon the signal received, may communicate any desired power output information to the controller 100. Thus, these embodiments may provide real time feedback which allows the control of the intensity of light emitted from the OLED 100. The detector 140 and the processor 120 may be powered by compact power supply, such as a battery pack 130, or by some other power source.

In some embodiments related to phototherapy, the LED device may further comprise a dosage component. A dosage component may be configured to provide a sufficient amount of light to activate a sufficient portion of a photosensitive compound to provide a therapeutic effect for treating a disease. For example, a dosage component may be a timer that is configured to deliver light from the device for an amount of time sufficient to deliver the appropriate light dosage. The timer may automatically stop the emission from the device once the appropriate light dosage has been delivered. The dosage component may also comprise a positioning component that positions the device so that emitted light is delivered to the appropriate area of a mammal body and is at an appropriate distance from the affected tissue to deliver an effective amount of light. The dosage component may be configured to work with a particular photosensitive compound, or may provide flexibility. For example, a physician, a veterinarian, or another appropriate medical practitioner may set the parameters of the dosage component for use by a patient outside of the practitioner's office, such as at the patient's home. In some embodiments, the device may be provided with a set of parameters for various photosensitive compounds to assist a medical practitioner in configuring the device.

In some embodiments, the device may further include a wireless transmitter electrically connected to an component of the apparatus generating treatment information, e.g., level of intensity, time of application, dosage amount, to communicate/transfer data to another external receiving device, like cell phone, PDA or to doctor's office. In some embodiments, the apparatus may further include an adhesive tape which may be used to attach the apparatus on the tissue surface so as to stabilize it on the target area.

For phototherapy and other applications, a wavelength convertor may be positioned in the device to receive at least a portion of light emitted from the organic light-emitting diode in a lower wavelength range, such as about 350 nm to less than about 600 nm, and convert at least a portion of the light received to light in a higher wavelength range, such as about 600 nm to about 800 nm. The wavelength convertor may be a powder, a film, a plate, or in some other form and, may comprise: yttrium aluminum garnet (YAG), alumina (Al₂O₃), yttria (Y₂O₃), titania (TiO₂), and the like. In some embodiments, the wavelength convertor may comprise at least one dopant which is an atom or an ion of an element such as Cr, Ce, Gd, La, Tb, Pr, Sm, Eu, etc.

In some embodiments, translucent ceramic phosphor is represented by a formula such as, but not limited to (A₁₋ₓEₓ)₃D₅O₁₂, (Y₁₋ₓEₓ)₃D₅O₁₂; (Gd₁₋ₓEₓ)₃D₅O₁₂; (La₁₋ₓEₓ)₃D₅O₁₂; (Lu₁₋ₓEₓ)₃D₅O₁₂; (Tb₁₋ₓEₓ)₃D₅O₁₂; (A₁₋ₓEₓ)₃Al₅O₁₂; (A₁₋ₓEₓ)₃Ga₅O₁₂; (A₁₋ₓEₓ)₃I₅O₁₂; (A₁₋ₓCeₓ)₃D₅O₁₂; (A₁₋ₓEuₓ)₃D₅O₁₂; (A₁₋ₓTbₓ)₃D₅O₁₂; (A₁₋ₓEₓ)₃Nd₅O₁₂; and the like. In some embodiments, the ceramic comprises a garnet, such as a yttrium aluminum garnet, with a dopant. Some embodiments provide a composition represented by the formula (Y₁₋ₓCeₓ)₃Al₅O₁₂. In any of the above formulas, A may be Y, Gd, La, Lu, Tb, or a combination thereof; D may be Al, Ga, In, or a combination thereof; E may be Ce, Eu, Tb, Nd, or a combination thereof; and x may be in the range of about 0.0001 to about 0.1, from about 0.0001 to about 0.05, or alternatively, from about 0.01 to about 0.03

### EXAMPLES

### Example 1

### General Synthetic Methods

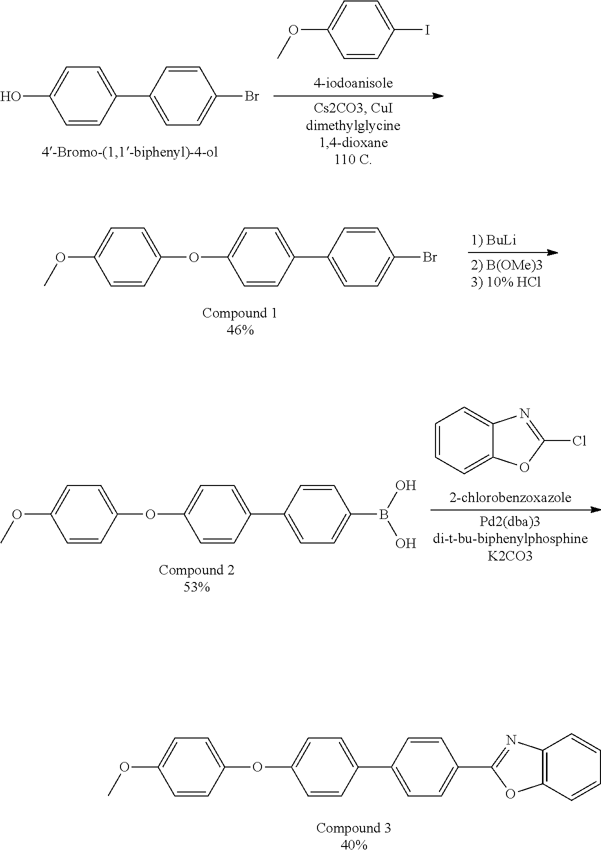

### Example 1.1.1

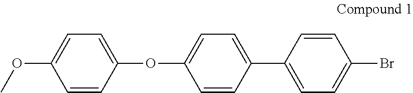

A mixture of 4′-Bromo-(1,1′-biphenyl)-4-ol (10.0 g, 40.1 mmol), 4-iodoanisole (18.72 g, 80.0 mmol), cesium carbonate (26.1 g, 80.2 mmol), copper iodide (760 mg, 4.0 mmol), dimethylglycine hydrochloride (1.68 g, 12.0 mmol), and anhydrous 1,4-dioxane (50 mL) was purged via freeze-pump-thaw method. The resulting mixture was heated to 110° C. overnight. After cooling, the resulting mixture was poured into ethyl acetate (300 mL), and stirred at 40° C. for 30 min; and the resulting solids were filtered off. The filtrate was dried under vacuum to give ivory solids. The ivory solids were washed with a mixture of ethyl acetate and methanol to give pure product (Compound 1); 6.5 g, 46% yield; confirmed by HNMR

### Example 1.1.2

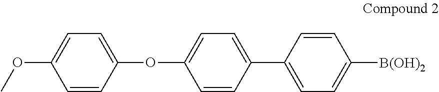

Compound 1(2 g, 5.63 mmol) was dissolved in anhydrous tetrahydrofuran (30 mL), and the resulting solution was cooled to −78° C. Butyllithium (3.43 mL of a 1.6 M solution, 5.5 mmol) was added dropwise and the solution was stirred at −78° C. for three hours. Trimethyl borate (0.572 mL, 5.5 mmol) was added slowly, and the resulting mixture was stirred for three hours at room temperature. Saturated ammonium chloride solution (45 mL) (alternatively, 10% HCl solution used) was added and the mixture was stirred overnight at room temperature. The desired product extracted with ethyl acetate (2×100 mL). The organic layer from the extraction was dried under vacuum. Precipitation in DCM/methanol gave white solids. The white solids were filtered and washed with methanol. The filtrate was dried to give relatively pure product (Compound 2); 1.2 g, 53% yield; relatively pure by HNMR.

### Example 1.1.3

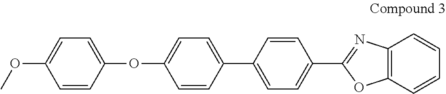

A mixture of Compound 2 (100 mg, 0.31 mmol), 2-chlorobenzoxazole (50 mg, 0.33 mmol), Pd(OAc)₂ (3.5 mg, 0.015 mmol), di-t-butyl-biphenylphosphine (9 mg, 0.03 mmol) and KF (54 mg, 0.93 mmol) in 1,4-dioxane (5 mL) was degassed then heated at 110° C. for 36 hours under argon. After cooling to room temperature, the mixture was poured into dichloromethane (100 mL). After filtration, the filtrate was loaded on silica gel column and purified by flash chromatography (hexanes/ethyl acetate 10:1 to 5:1). A white solid was obtained (40 mg, 40%) as the desired product (Compound 3), which was confirmed by LCMS (calculated for C₂₆H₂₀NO₃ (M+H): 394; found m/e=394) and HNMR.

### Example 1.2

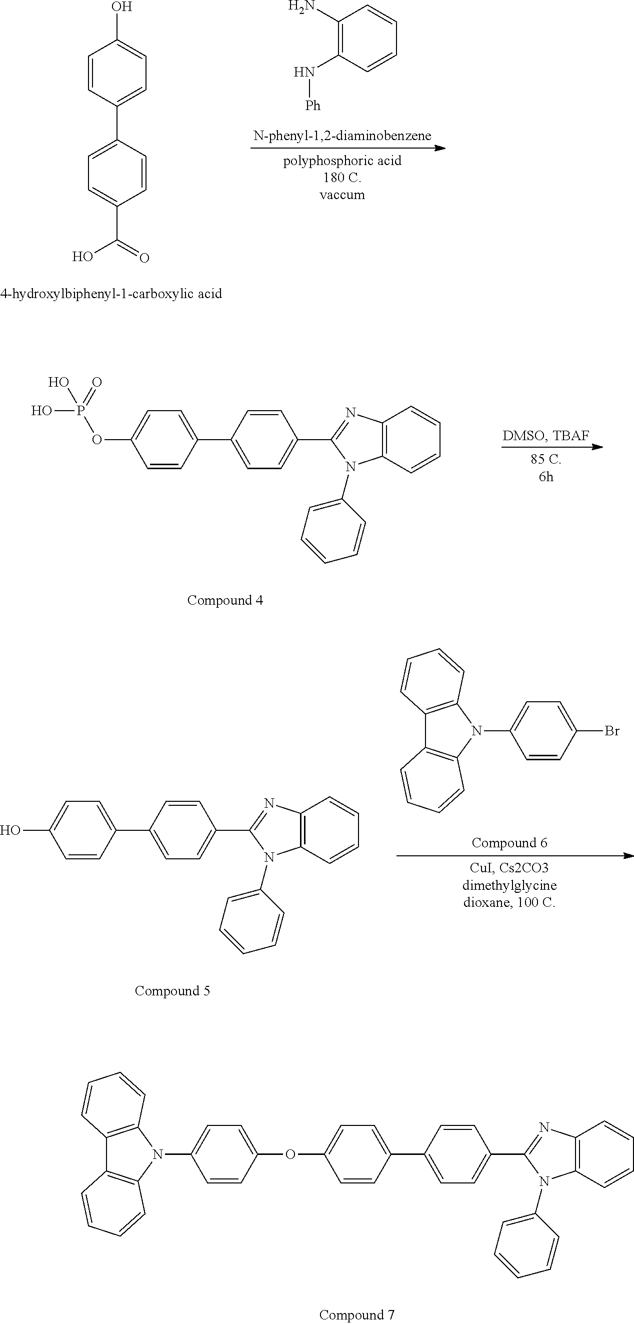

### Example 1.2.1

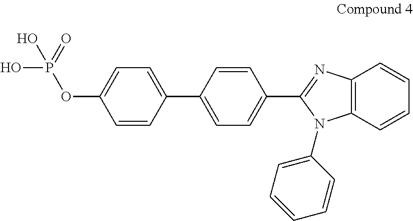

A mixture of 4-hydroxylbiphenyl-1-carboxylic acid (2.14 g, 10 mmol) and N-phenyl-1,2-diaminobenzene (1.84 g, 10 mmol) in polyphosphoric acid (10 ml) was degassed by vacuum then heated at 180° C. at 10 torr overnight. After cooling to room temperature, the mixture was poured into water. Filtration and washing with water gave a dark solid (6.8 g, 84%) as the desired product (Compound 4), which was confirmed by LCMS (calculated for C₂₅H₁₈N₂O₄P (M−H): 441, found m/e: 441).

### Example 1.2.2

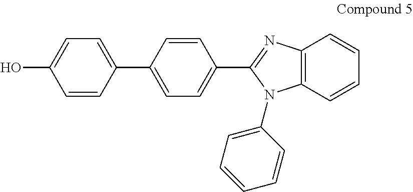

To a solution of Compound 4 (3.6 g, 8 mmol) in DMSO (20 mL), was added tetrabutylammonium fluoride (1M in THF, 18 mL). The mixture was heated at 85° C. for 6 hours. Water (50 mL) was added to the hot solution, followed by 5 mL concentrated HCl. The mixture was stirred for 5 min and allowed to cool down to room temperature. Filtration and drying under vacuum at 110° C. overnight afford a black solid (1.6 g, 55%) as desired product (Compound 5), which was confirmed by LCMS (calculated for C₂₅H₁₇N_{2O} (M−H): 361; found m/e=361).

### Example 1.2.3

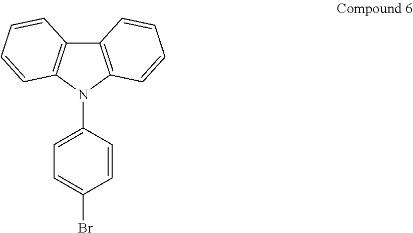

A mixture of carbazole (7.0 g, 42.2 mmol), 4-bromo-iodobenzene (17.9 g, 63.3 mmol), copper (13.6 g, 214 mmol), 18-crown-6 (4.36 g, 16.48 mmol), potassium carbonate (29.5 g, 214 mmol), and anhydrous N,N-dimethylformamide (50 mL) was degassed for 30 minutes. The mixture was heated to 150° C. overnight under argon. After cooling, the mixture was poured into DCM (400 mL) and the subsequent mixture was filtered. The filtrate was concentrated under vacuum, and hexane was added to precipitate out 18-crown-6, which was filtered off. The filtrate was loaded on silica gel. A flash column (silica, 100% hexane) and reprecipitation in DCM/hexanes yielded 9.44 g of relatively pure product (Compound 6) (white crystals) in 70% yield; confirmed by HNMR.

### Example 1.2.4

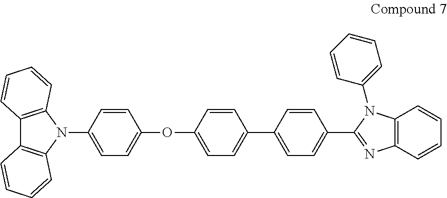

A mixture of Compound 6 (1.09 g, 3.40 mmol), Compound 5 (1.24 g, 3.42 mmol), cesium carbonate (2.23 g, 6.84 mmol), copper iodide (65 mg, 0.342 mmol), dimethylglycine hydrochloride (95 mg, 0.684 mmol) and anhydrous 1,4-dioxane (30 mL) was degassed for 30 minutes. The mixture was heated to 120° C. overnight under argon. After cooling, the resulting mixture was poured into DCM (250 mL) and filtered. The resulting filtrate was loaded onto silica gel. The resulting effluent from the flash column (silica, 5% to 50% ethyl acetate in hexanes gradient) gave an orange solid. Precipitation of the orange impurity was accomplished using DCM and methanol. The impurity was filtered off, and the filtrate containing the product was dried under vacuum. After drying, 270 mg of product (Compound 7) (pale orange powder, 13% yield) was isolated; pure by HNMR.

### Example 1.3

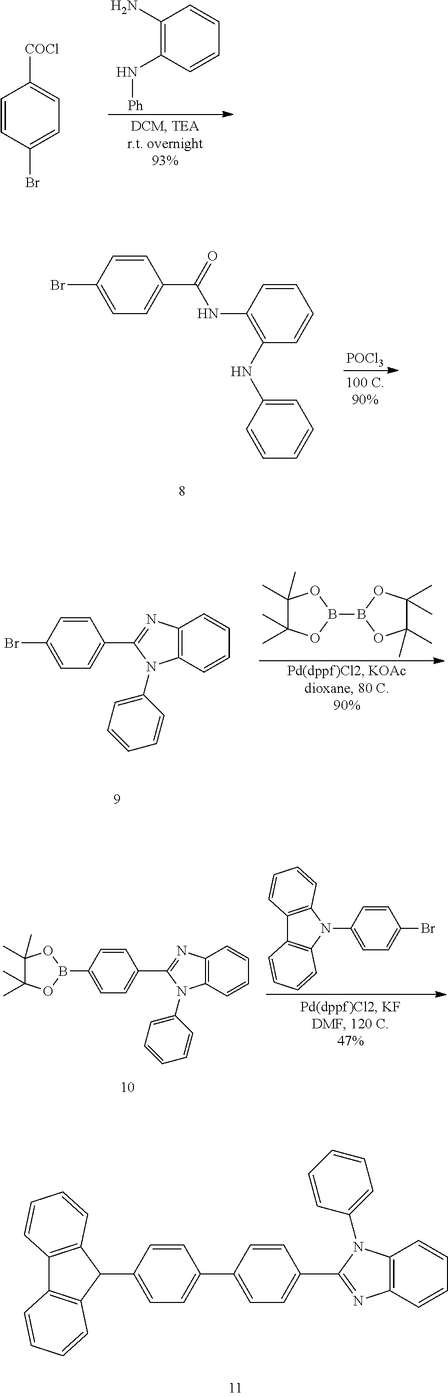

### Example 1.3.1

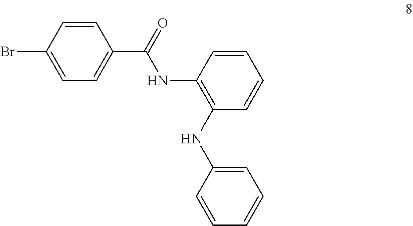

4-Bromo-N-(2-(phenylamino)phenyl)benzamide (8): To a solution of 4-bromo-benzoyl chloride (11 g, 50 mmol) in anhydrous dichloromethane (DCM) (100 ml), was added N-phenylbenzene-1,2-diamine (10.2 g, 55 mmol), then triethylamine (17 ml, 122 mmol) slowly. The whole mixture was then stirred at room temperature (r.t.) overnight. Subsequent filtration gave a white solid (Compound 8) (6.5 g). The filtrate was worked up with water (300 ml), and then extracted with DCM (300 ml) three times. The resulting organic phase was collected and dried over MgSO₄, concentrated and recrystallized in DCM/hexanes to give another portion of white solid (Compound 8) (10.6 g). The total amount of product (Compound 8) was 17.1 g, in 93% yield.

### Example 1.3.2

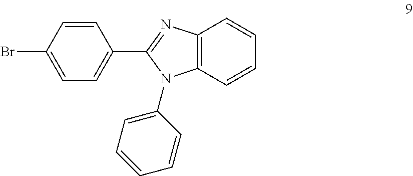

2-(4-bromophenyl)-1-phenyl-1H-benzo[d]imidazole (9): To a suspension of amide 1 (9.6 g, 26 mmol) in anhydrous 1,4-dioxane (100 mL) was added phosphorous oxychloride (POCl₃) (9.2 mL, 100 mmol) slowly. The whole was then heated at 100° C. overnight. After cooling to r.t., the mixture was poured into ice (200 g) with stirring. Filtration, followed by recrystallization in DCM/hexanes gave a pale grey solid (Compound 9) (8.2 g, in 90% yield).

### Example 1.3.3

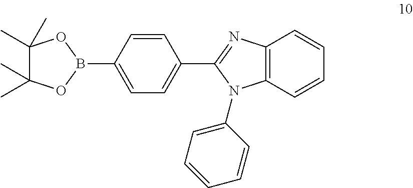

1-phenyl-2-(4-(4,4,5,5-tetramethyl-1,3,2-dioxaborolan-2-yl)phenyl)-1H-benzo[d]imidazole (10): A mixture of Compound 9 (0.70 g, 2 mmol), bis(pinacolate)diborane (0.533 g, 2.1 mmol), 1,1′-Bis(diphenylphosphino)ferrocene]dichloropalladium (Pd(dppf)Cl₂) (0.060 g, 0.08 mmol) and anhydrous potassium acetate (0.393 g, 4 mmol) in 1,4-dioxane (20 mL) was heated at 80° C. under argon overnight. After cooling to r.t., the whole mixture was diluted with ethyl acetate (80 mL) then filtered. The solution was absorbed on silica gel, then purified by column chromatography (hexanes/ethyl acetate 5:1 to 3:1) to give a white solid (Compound 10) (0.64 g, in 81% yield).

### Example 1.3.4

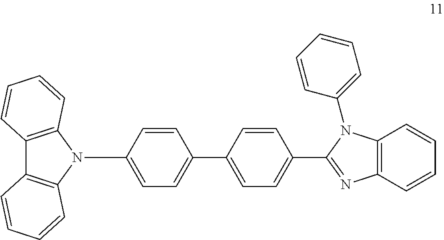

9-(4′-(1-phenyl-1H-benzo[d]imidazol-2-yl)biphenyl-4-yl)-9H-carbazole (11): A mixture of Compound 10 (1.41 g, 3.56 mmol), 9-(4-bromophenyl)-9H-carbazole (1.15 g, 3.56 mmol), Pd(dppf)Cl2 (100 mg, 0.14 mmol) and KF (0.619 g, 10.7 mmol) in anhydrous DMF (20 mL) was heated at 120° C. under argon overnight. After cooling to r.t., ethyl acetate (200 mL) was added and the whole was stirred for 15 min. The resulting mixture was filtered. The solid was collected and dissolved in DCM (200 mL), which was filtered, concentrated and recrystallized to give a white solid (Compound 11) (550 mg).

The filtrate from the first separation was absorbed on silica gel, then purified by column chromatography (hexanes/ethyl acetate 7:1 to 3:1) to give a pale yellow solid (Compound 11) (300 mg). NMR shows both of the two solids are desired product (Compound 11), with total amount of 850 mg, in 47% yield.

### Example 1.4

### Overall Scheme

Error! Objects cannot be created from editing field codes.

### Example 1.4.1

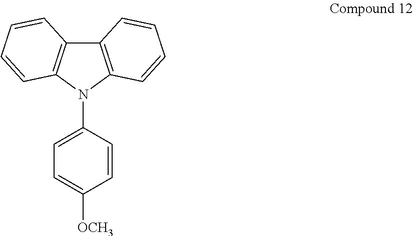

9-(4-methoxyphenyl)-9H-carbazole (Compound 12): A mixture of carbazole (10.0 g, 60.2 mmol), 4-iodoanisole (21.1 g, 90.4 mmol), copper powder (28.58 g, 450 mmol), 18-crown-6 (9.33 g, 35.3 mmol), and potassium carbonate (62.1 g, 450 mmol) was degassed in dimethylformamide (anhydrous, 100 mL) for 45 minutes. The resulting mixture was heated to 150° C. overnight under argon. After cooling, the mixture was poured into DCM (500 mL). Then, the remaining copper and salts were filtered off. The resulting filtrate washed with water (2×200 mL). The organic layer was collected, dried over sodium sulfate, and then loaded onto silica gel. A flash column (gradient of 3-5% ethyl acetate in hexanes) and reprecipitation from DCM/hexanes gave 11.71 g (71% yield) of product (Compound 12); confirmed pure by HNMR.

### Example 1.4.2

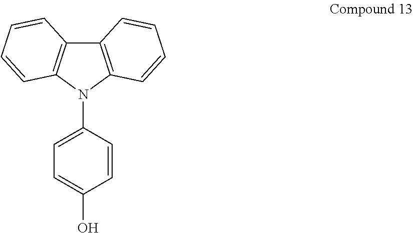

4-(9H-carbazol-9-yl)phenol (Compound 13): Compound 12 (11.63 g, 42.6 mmol) was dissolved in DCM and the solution was cooled to −77° C. Boron tribromide (45 mL of a 1M solution) was added dropwise to the cold solution. The solution was stirred overnight under argon while slowly warming to room temperature. LCMS showed a single peak with desired mass (M⁻=528). Methanol (100 mL) was added to reaction mixture; and stirred for 30 minutes. The whole was loaded onto silica gel. A flash column (gradient of 10-20% ethyl acetate in hexanes) gave 10.94 g of product (Compound 13) (99% yield), confirmed pure by HNMR.

### Example 1.4.3

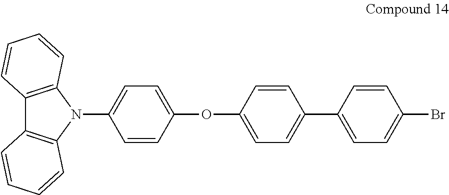

9-(4-(4′-bromobiphenyl-4-yloxy)phenyl)-9H-carbazole (Compound 14): A mixture of the Compound 13 (10.0 g, 8.3 mmol), 4,4′-dibromobiphenyl (24.1 g, 77.2 mmol), cesium carbonate (25.2 g, 77.2 mmol), copper iodide (700 mg, 3.86 mmol), and dimethylglycine hydrochloride (1.62 g, 11.6 mmol) was degassed in 1,4-dioxane (anhydrous, 100 mL) for 45 minutes. The mixture was heated to 120° C. overnight under argon. After cooling, the mixture was poured into DCM (300 mL) the subsequent mixture was washed with water and brine. The organic layer was collected, dried over sodium sulfate, and loaded onto silica gel. A plug using 10% ethyl acetate in hexanes was used to remove baseline impurities. A subsequent flash column (5% toluene in hexanes), and precipitation from DCM/methanol gave 11.17 g of product (Compound 14) (59% yield), confirmed pure by HNMR.

### Example 1.4.4

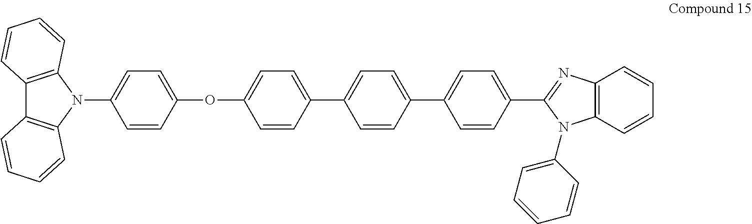

A mixture of the Compound 14 (1.85 g, 3.79 mmol), 1-phenyl-2-(4-(4,4,5,5-tetramethyl-1,3,2-dioxaborolan-2-yl)phenyl)-1H-benzo[d]imidazole (1.5 g, 3.79 mmol), 1,1′-Bis(diphenylphosphino)ferrocene]dichloropalladium (139 mg, 0.190 mmol), and potassium fluoride (661 mg, 11.4 mmol) was degassed in dimethylformamide (anhydrous, 30 mL) for 30 minutes. The mixture was heated to 80° C. overnight under argon. After cooling, the mixture was poured into DCM (200 mL) and washed with water and brine. The organic layer was collected, dried over sodium sulfate, and loaded onto silica gel. A flash column (20% ethyl acetate in hexanes) and recrystallization from DCM/methanol gave 1.83 g (71% yield) of product (Compound 15); confirmed pure by HNMR.

### Example 1.5

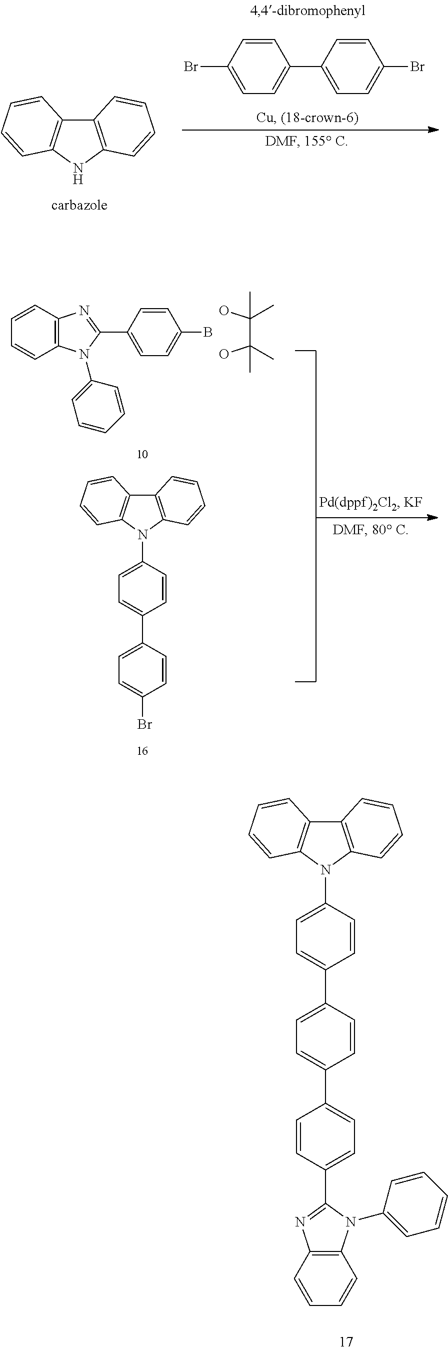

### Example 1.5.1

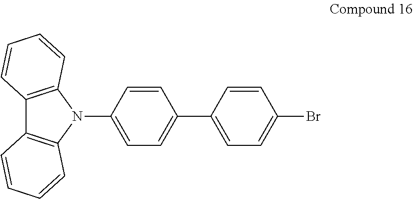

9-(4′-bromobiphenyl-4-yl)-9H-carbazole: A mixture of carbazole (300 mg, 1.81 mmol), 4,4′-dibromobiphenyl (846 mg, 2.71 mmol), copper (344 mg, 5.43 mmol), 18-crown-6 (187 mg, 0.71 mmol), potassium carbonate (750 mg, 5.43 mmol), and anhydrous N,N-dimethylformamide (10 mL) was degassed for 30 minutes. The mixture was heated to 155° C. for 66 hours under argon. After cooling, the mixture was poured into DCM (400 mL) and the subsequent mixture was filtered. The filtrate was loaded on silica gel. A flash column (silica, 10% DCM in hexane) and reprecipitation in DCM/hexanes yielded 304 mg (42% yield) of pure product; confirmed by HNMR.

### Example 1.5.2

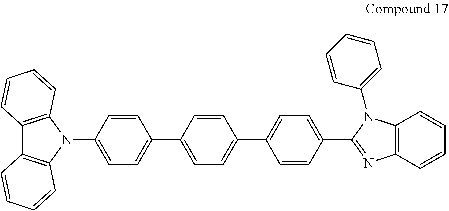

A mixture of Compound 16 (250 mg, 0.63 mmol), 1-phenyl-2-(4-(4,4,5,5-tetramethyl-1,3,2-dioxaborolan-2-yl)phenyl)-1H-benzo[d]imidazole (Compound 10) (250 mg, 0.63 mmol), [1,1′-Bis(diphenylphosphino)ferrocene]dichloropalladium(II) (23 mg, 0.03 mmol), potassium fluoride (110 mg, 1.89 mmol), and dimethylformamide (anhydrous, 15 mL) was degassed for 20 minutes. The mixture was then heated to 80° C. overnight under argon. After cooling, the mixture was poured into DCM (200 mL), and solids were filtered off. The resulting filtrate was washed with water (2×100 mL), dried over sodium sulfate, and then loaded onto silica gel. A flash column (gradient of 3-10% ethyl acetate in DCM) and recrystallization using DCM/hexanes gave 170 mg (46% yield) of material (Compound 17); confirmed pure by HNMR.

### Example 1.6

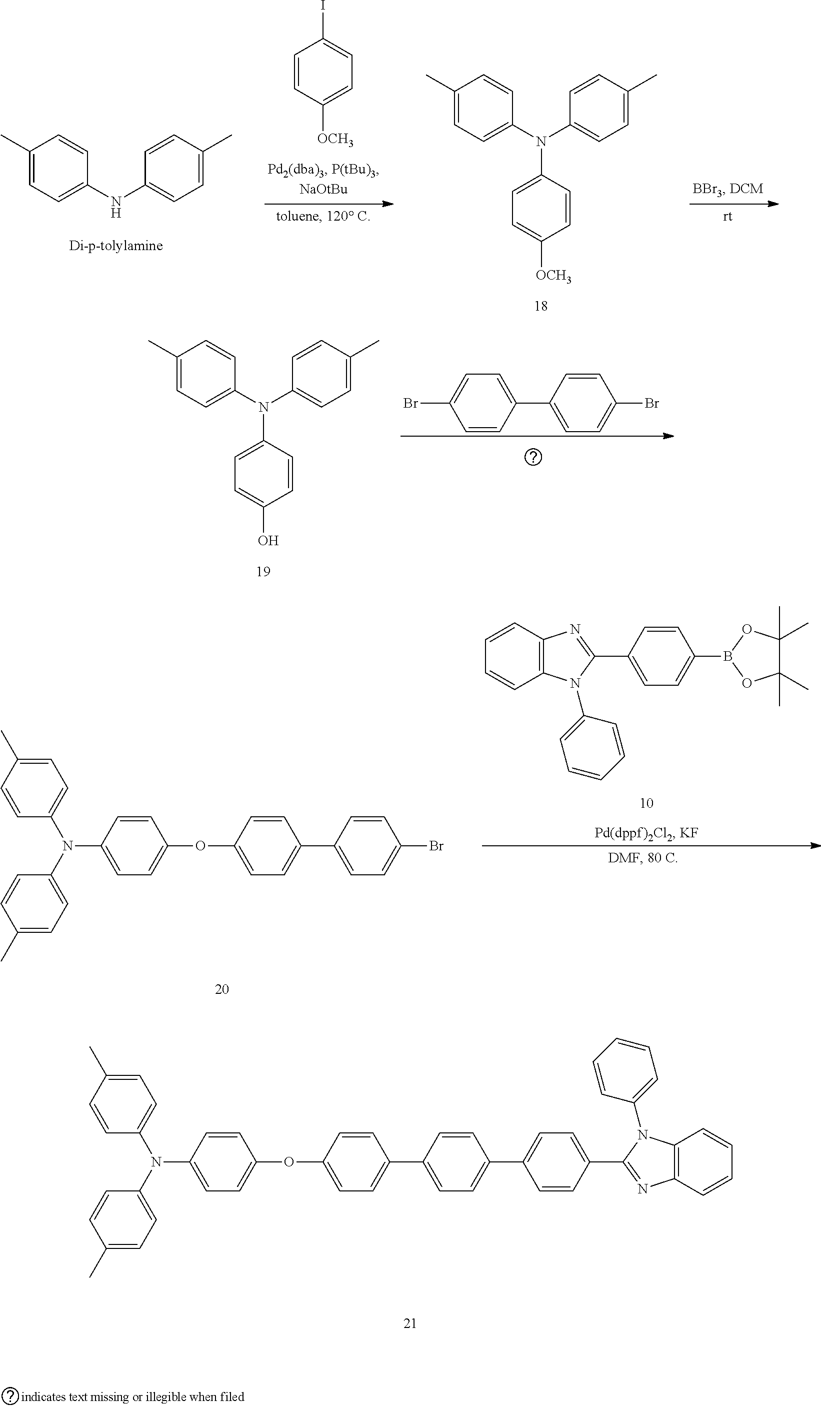

### Example 1.6.1

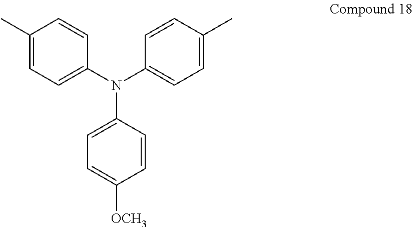

4-Methoxy-N,N-di-p-tolylaniline (Compound 18): A mixture of tris(dibenzylideneacetone)dipalladium (60 mg, catalytic), and tri-tert-butyl phosphine (5 ml of a 10% solution in hexanes) was degassed in toluene (anhydrous, 60 mL) for 20 minutes. Di-p-tolylamine (4.0 g, 20.3 mmol), and 4-iodoanisole (11.88 g, 50.8 mmol) were added and degassing continued 15 minutes. Sodium tert-butoxide (2.4 g, 25 mmol) was added, and mixture was further degassed for 10 minutes. The whole mixture was heated overnight at 120° C. under argon. After cooling, the mixture was poured into ethyl acetate and washed with water (2×200 mL). The organic layer was collected and dried over sodium sulfate, then loaded onto silica gel. A flash column (gradient of 2-3% ethyl acetate in hexanes) gave 3.26 g of material (Compound 18) (53% yield); confirmed pure by HNMR.

### Example 1.6.2

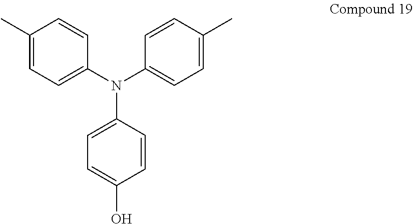

4-(di-p-tolylamino)phenol (Compound 19): Compound 18 (3.05 g, 10.1 mmol) was dissolved in DCM (anhydrous, 50 mL) and the solution was cooled to −77° C. Boron tribromide (12 mL of a 1M solution) was added dropwise to the cold solution. The whole mixture was stirred and slowly warmed to room temperature overnight under argon. LCMS showed a single desired mass of 288 (M⁻). The mixture was poured into methanol (200 mL) and stirred for 45 minutes. The mixture was then concentrated under vacuum, then DCM (100 mL) was added. The solution was washed with water (2×200 mL). The organic layer was collected, dried over sodium sulfate, and concentrated under vacuum. Precipitation of the organic layer using hexanes gave 2.44 g of material (Compound 19) (84% yield); confirmed pure by HNMR.

### Example 1.6.3

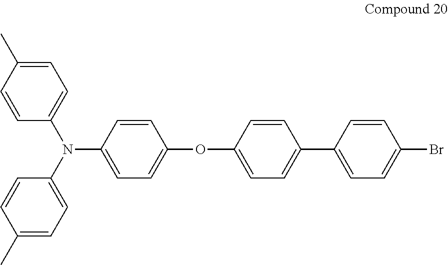

4-(4′-bromobiphenyl-4-yloxy)-N,N-di-p-tolylaniline (Compound 20): A mixture of the Compound 19 (2.4 g, 8.3 mmol), 4,4′-dibromobiphenyl (1.25 g, 4.0 mmol), cesium carbonate (5.41 g, 16.6 mmol), copper iodide (158 mg, 0.83 mmol), and dimethylglycine hydrochloride (348 mg, 2.49 mmol) was degassed in 1,4-dioxane (anhydrous, 40 mL) for 45 minutes. The mixture was heated to 120° C. overnight under argon. After cooling, the mixture was poured into DCM (300 mL), and the subsequent mixture was washed with water and brine. The organic layer was collected, dried over sodium sulfate, and loaded onto silica gel. A flash column (gradient of 5-20% DCM in hexanes), and reprecipitation from DCM/hexanes gave 760 mg of product (Compound 20) (37% yield), confirmed pure by HNMR.

### Example 1.6.4

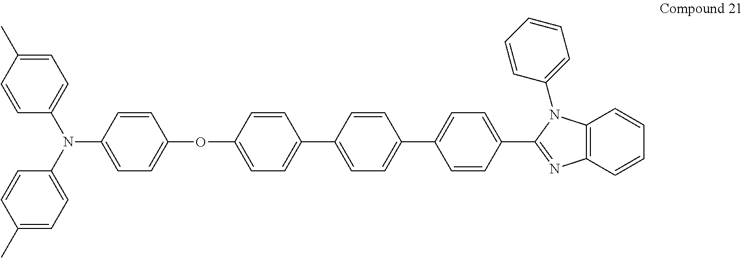

A mixture of Compound 20 (656 mg, 1.26 mmol), 1-phenyl-2-(4-(4,4,5,5-tetramethyl-1,3,2-dioxaborolan-2-yl)phenyl)-1H-benzo[d]imidazole (500 mg, 1.26 mmol), 1,1′-Bis(diphenylphosphino)ferrocene]dichloropalladium (100 mg, catalytic), and potassium fluoride (220 mg, 3.8 mmol) was degassed in dimethylformamide (anhydrous, 8 mL) for 30 minutes. Mixture was heated to 80° C. overnight under argon. After cooling, the mixture was poured into water (200 mL). Product was extracted with DCM (2×150 mL). The organic layer collected, dried over sodium sulfate, and loaded onto silica gel. A flash column (gradient of 15-45% ethyl acetate in hexanes) and reprecipitation from DCM/methanol gave 400 mg (45% yield) of product; confirmed pure by HNMR.

### Example 1.7

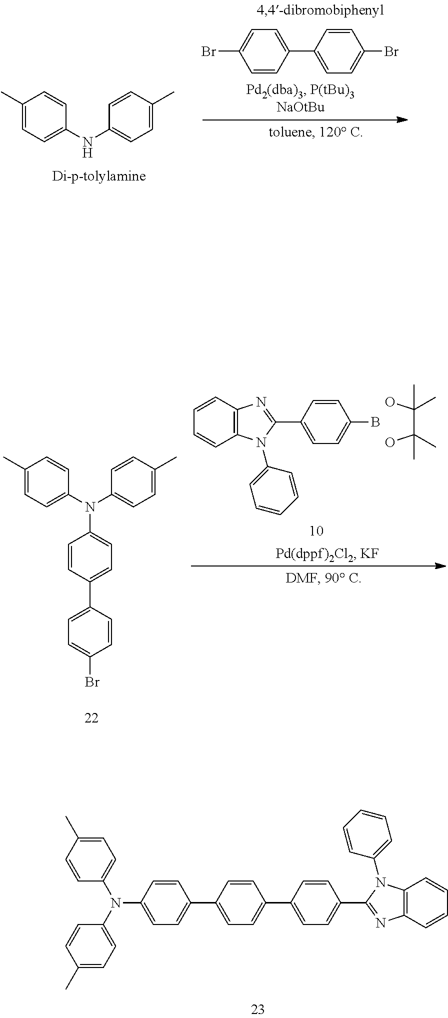

### Example 1.7.1

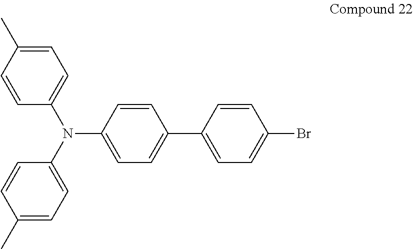

4′-bromo-N,N-di-p-tolylbiphenyl-4-amine (Compound 22): A mixture of tris(dibenzylideneacetone)dipalladium (353 mg, 0.385 mmol), and tri-tert-butyl phosphine (3.11 g of a 10% solution in hexanes) was degassed in toluene (anhydrous, 50 mL) for 20 minutes. Di-p-tolylamine (3.00 g, 15.2 mmol), and 4,4′-dibromobiphenyl (4.80 g, 15.4 mmol) were added and the mixture was further degassed for 15 minutes. Sodium tert-butoxide (2.4 g, 25 mmol) was added, and mixture was further degassed for 10 minutes. The whole was heated overnight at 120° C. under argon. After cooling, the mixture was poured into DCM, and solids were filtered off. Filtrate was washed with water and brine. The organic layer was collected and dried over sodium sulfate, then loaded onto silica gel. A flash column (gradient of 2-20% ethyl acetate in hexanes) gave 190 mg of material (3% yield); confirmed pure by HNMR.

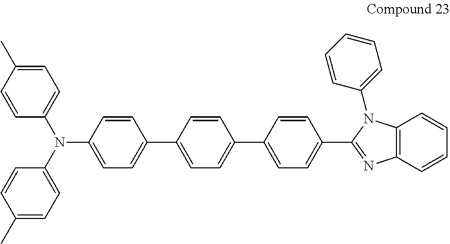

### Example 1.7.2

A mixture of Compound 22 (170 mg, 0.397 mmol), 1-phenyl-2-(4-(4,4,5,5-tetramethyl-1,3,2-dioxaborolan-2-yl)phenyl)-1H-benzo[d]imidazole (173 mg, 0.436 mmol), 1,1′-Bis(diphenylphosphino)ferrocene]dichloropalladium (15 mg, 0.02 mmol), and potassium fluoride (70 mg, 1.2 mmol) was degassed in dimethylformamide (anhydrous, 15 mL) for 30 minutes. Mixture was heated to 90° C. overnight under argon. After cooling, the mixture was poured into water and filtered. The solids were dissolved in DCM (50 mL) and solution was washed with water and brine. To filtrate from first filtration: filtrate was extracted with DCM (2×100 mL). All organic phases were combined, dried over sodium sulfate, and loaded onto silica gel. A flash column (gradient of 10-20% ethyl acetate in hexanes) and reprecipitation in DCM/methanol gave 146 mg (59% yield) of product (Compound 23); confirmed by HNMR.

### Example 2

### OLED Device Configuration and Performance

### Example 2.1

Fabrication of light-emitting device (Device A): the ITO coated glass substrates were cleaned by ultrasound in acetone, and consecutively in 2-propanol, baked at 110° C. for 3 hours, followed by treatment with oxygen plasma for 5 min. A layer of PEDOT: PSS (Baytron P purchased from H.C. Starck) was spin-coated at 3000 rpm onto the pre-cleaned and O₂-plasma treated (ITO)-substrate and annealed at 180° C. for 10 min, yielding a thickness of around 40 nm. The substrate is then transferred into a glove-box hosted vacuum deposition system at a pressure of 10⁻⁷ torr (1 torr=133.322 Pa) where hole transporting layer 4,4′-bis[N-(naphthyl)-N-phenyl-amino]biphenyl (NPD) was vacuum deposited at a rate of about 0.1 nm/s rate, yielding a 30 nm thick film. The emissive material, bis[(1-phenylisoquinolinato-N,C2′)]iridium (III) (acetylacetonate) (Ir(piq)₂acac) (9 wt %) was then co-deposited with Compound 23 at about 0.01 nm/sec and about 0.1 nm/s, respectively, to make the appropriate doping ratio and to form a 30 nm thick emissive layer, followed by deposition of a 40 nm thick layer of 1,3,5-tris(N-phenylbenzimidizol-2-yl)benzene (TPBI). LiF and Al were then deposited successively at deposition rates of 0.005 and 0.2 nm/s, respectively. Each individual device has areas of 0.14 cm². Spectra is measured with an ocean optics HR4000 spectrometer and I-V light output measurements is taken with a Keithley 2400 SourceMeter and Newport 2832-C power meter and 818 UV detector. All device operation is carried out inside a nitrogen-filled glove-box.

### Example 2.2

Another device (Device B) was constructed in accordance to Example 2.1, except that Compound 17 is used instead of Compound 23 as a host in the emissive layer.

### Example 3

### Device Performance

The comparison data between the Device A and Device B is listed in the following table. It shows clearly that the Device A (Compound 23 as host) outperforms Device B (use Compound 17 as host).

| | | | LE | PE | |
|---|---|---|---|---|---|
| | V(V) | I (mA) | (cd/A) | (Im/W) | EQE |
| Device A | 3.76 | 0.95 | 9.28 | 7.75 | 6.80% |
| Device B | 3.73 | 1.55 | 5.16 | 4.34 | 3.68% |

### Example 4

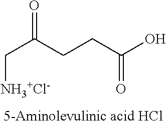

5-Aminolevulinic acid HCl (20% topical solution, available as LEVULAN® KERASTICK® from DUSA® Pharmaceuticals) is topically applied to individual lesions on a person suffering from actinic keratoses. About 14-18 hours after application, the treated lesions are illuminated with a red light emitting OLED device constructed as set forth in Example 3, Device A.

After the treatment, the number or severity of the lesions is anticipated to be reduced. The treatment is repeated as needed.

### Example 5

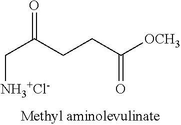

Methyl aminolevulinate (16.8% topical cream, available as METVIXIA® Cream from GALERMA LABORATORIES, Fort Worth, Tex., USA) is topically applied to individual lesions on a person suffering from actinic keratoses. The excess cream is removed with saline, and the lesions are illuminated with the red light emitting OLED constructed as set forth in Example 3, Device A.

Nitrile gloves are worn at all times during the handling of methyl aminolevulinate. After the treatment, it is anticipated that the number or severity of the lesions is reduced. The treatment is repeated as needed.

### Example 6

Verteporphin is intravenously injected, over a period of about 10 minutes at a rate of about 3 mL/min, to a person suffering from age-related macular degeneration. The verteporphin (7.5 mL of 2 mg/mL reconstituted solution, available as Visudyne® from Novartis) is diluted with 5% dextrose to a volume of 30 mL using a sufficient quantity of the reconstituted verteporphin so that the total dose injected is about 6 mg/m² of body surface.

About 15 minutes after the start of the 10 minute infusion of verteporphin, the verteporphin is activated by illuminating the retina with a red light emitting OLED device as set forth in Example 3, Device A.

After treatment, the patient's vision is anticipated to be stabilized. The treatment is repeated as needed.

### Example 7

Verteporphin is intravenously injected, over a period of about 10 minute at a rate of about 3 mL/min, to a person suffering from pathological myopia. The verteporphin (7.5 mL of 2 mg/mL reconstituted solution, available as Visudyne® from Novartis) is diluted with 5% dextrose to a volume of 30 mL using a sufficient quantity of the reconstituted verteporphin so that the total dose injected is about 6 mg/m² of body surface.

About 15 minutes after the start of the 10 minute infusion of verteporphin, the verteporphin is activated by illuminating the retina with a red light emitting OLED device as set forth in Example 3, Device A.

After treatment, the patient's vision is anticipated to be stabilized. The treatment is repeated as needed.

### Example 8

Verteporphin is intravenously injected, over a period of about 10 minutes at a rate of about 3 mL/min, to a person suffering from presumed ocular histoplasmosis. The verteporphin (7.5 mL of 2 mg/mL reconstituted solution, available as Visudyne® from Novartis) is diluted with 5% dextrose to a volume of 30 mL using a sufficient quantity of the reconstituted verteporphin so that the total dose injected is about 6 mg/m² of body surface.

About 15 minutes after the start of the 10 minute infusion of verteporphin, the verteporphin is activated by illuminating the retina with a red light emitting OLED device as set forth in Example 3, Device A.

After treatment, the patient's vision is anticipated to be stabilized. The treatment is repeated as needed.

It will be understood by those of skill in the art that numerous and various modifications can be made without departing from the spirit of the present invention. Therefore, it should be clearly understood that the forms of the present invention are illustrative only and are not intended to limit the scope of the present invention.

What is claimed is:

1. A light-emitting device for use in phototherapy comprising:
a light-emitting layer comprising a compound represented by a formula: 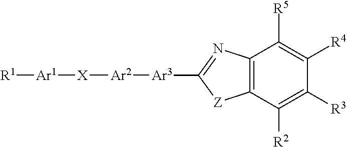
wherein R¹ is a C₁₋₁₀O₁₄ ether attaching at an oxygen atom or —R⁷—NR⁸R⁹;
wherein R⁷ is a single bond, optionally substituted C₆₋₁₀ aryloxy, or optionally substituted C₆₋₁₀ aryl; and
R⁸ and R⁹ are independently optionally substituted C₆₋₁₀ aryl, wherein
R⁸ and R⁹ optionally link together form a third ring comprising N;
Ar¹ and Ar² are independently aryl having 0, 1, or 2 substituents independently selected from C₁₋₃ alkyl, F, and Cl;
X is O or a single bond;
Ar³ is aryl having 0, 1, or 2 substituents independently selected from C₁₋₃ alkyl, F, and Cl; or Ar³ is a single bond;
Z is independently NR⁶O, or S, wherein R⁶ is optionally substituted phenyl, optionally substituted —CH₂-phenyl, or optionally substituted (4-halophenyl)methyl; and
R², R³, R⁴, and R⁵ are independently H, optionally substituted C₆₋₃₀ aryl, C₁₋₁₀ alkyl, or C₁₋₁₀ alkoxy; and
wherein the device is configured to emit a therapeutically effective amount of light to a mammal.

2. The device of claim 1 wherein R¹ is methoxy,

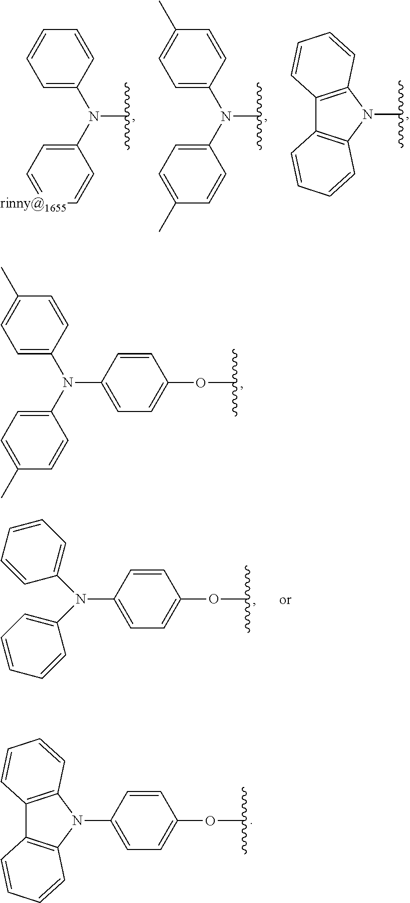

3. The device of claim 1, wherein Ar¹, Ar², and Ar³ are optionally substituted p-interphenylene.

4. The device of claim 1, wherein X is O.

5. The device of claim 1, wherein X is a single bond.

6. The device of claim 5, wherein Ar³ is a single bond.

7. The device of claim 5, wherein Ar³ is aryl having 0, 1, or 2 substituents independently selected from C₁₋₃ alkyl, F, and Cl.

8. The device of claim 1, wherein Z is O.

9. The device of claim 1, wherein Z is NR⁶ and R⁶ is optionally substituted phenyl.

10. The device of claim 1, wherein the compound is selected from:

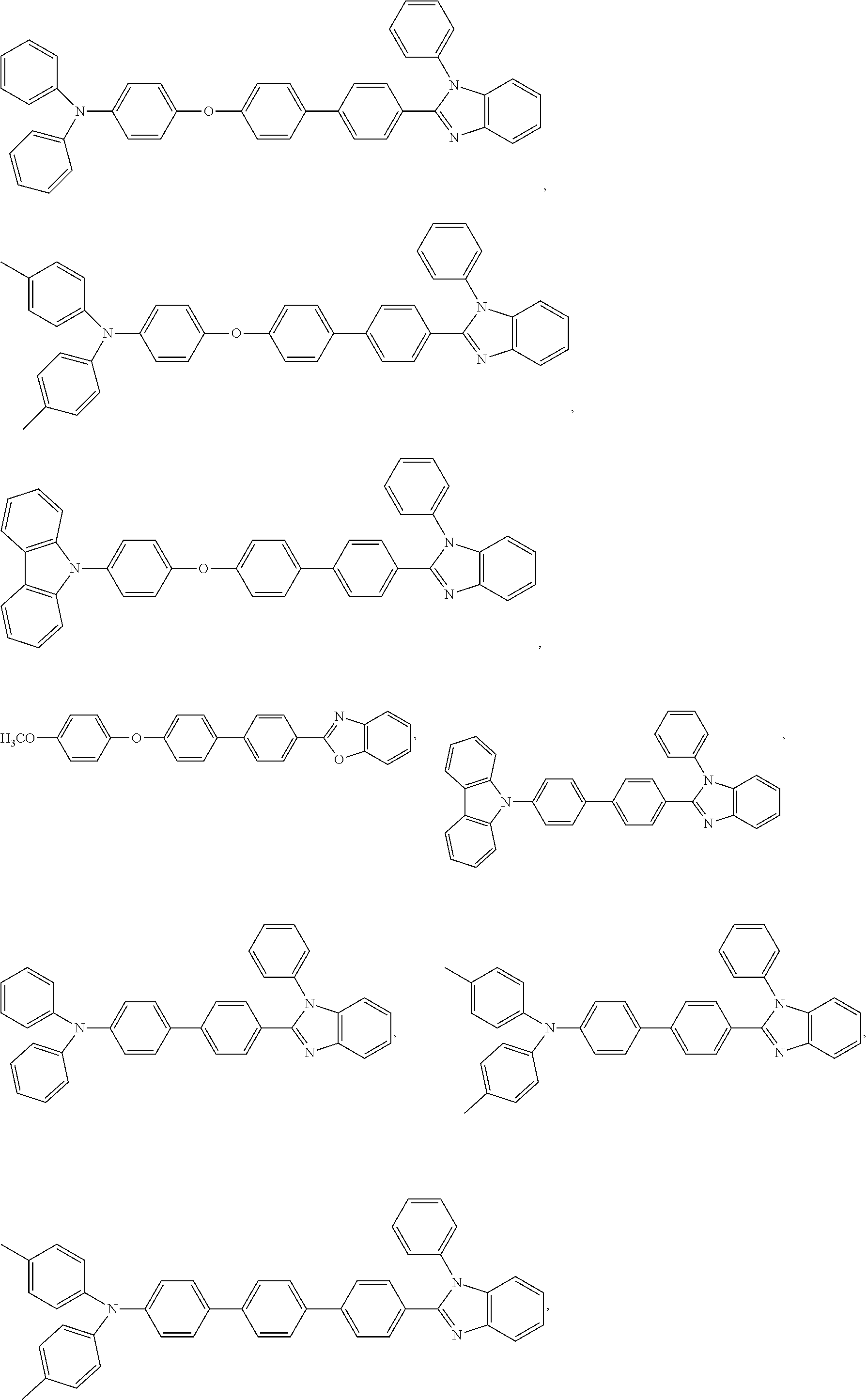

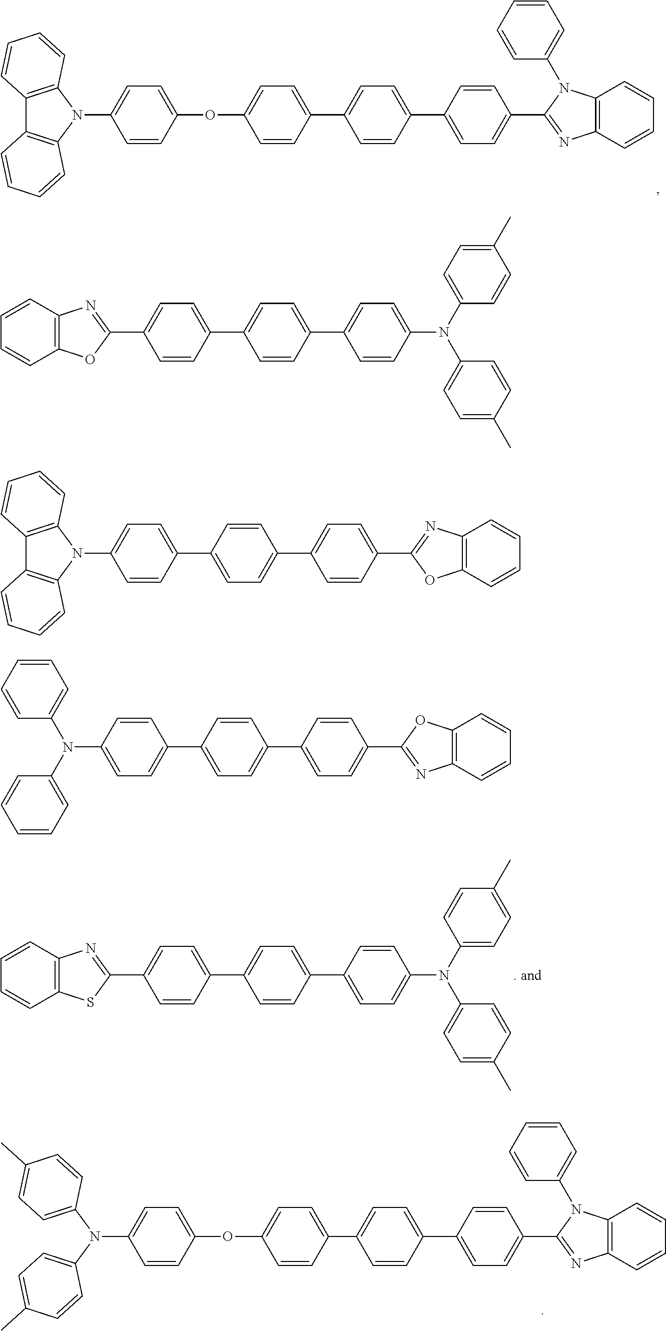

11. The device of claim 1, wherein the device is configured to emit light of a wavelength that can activate at least a portion of a photosensitive compound which has been administered to a tissue of a mammal; and
wherein the device further comprises a dosage component configured to provide a sufficient amount of light to activate a sufficient portion of the photosensitive compound to provide a therapeutic effect for treating a disease.

12. A method of carrying out phototherapy comprising:
exposing at least a portion of a tissue of a mammal to light from a device of claim 1.

13. The method of claim 12, further comprising administering a photosensitive compound to the tissue, and wherein at least a portion of the photosensitive compound is activated by exposing the portion of the tissue to light from the device.

14. A method of treating a disease, comprising:
administering a photosensitive compound to a tissue of a mammal in need thereof; and
exposing at least a portion of the tissue to light from a device of claim 1;
wherein at least a portion of the photosensitive compound is activated by at least a portion of the light from the device to which the tissue is exposed, to thereby treat the disease.

15. The method of claim 14, wherein activating the photosensitive compound produces singlet oxygen.

16. The method of claim 14, wherein the photosensitive compound is 5-aminolevulinic acid, verteporfin, zinc phthalocyanine, or pharmaceutically acceptable salts thereof.

17. The method of claim 14, wherein the disease is cancer.

18. The method of claim 14, wherein the disease is a microbial infection.

19. The method of claim 14, wherein the disease is a skin condition.

20. The method of claim 14, wherein the disease is an eye condition.

21. A phototherapy system comprising:
a device according to claim 1; and
a photosensitive compound;
wherein the photosensitive compound is suitable for administration to a tissue of a mammal in need of phototherapy; and
wherein the device is configured to emit light of a wavelength which can activate at least a portion of the photosensitive compound when it is in the tissue.
